(19) United States
(12) Patent Application Publication
(10) Pub. No.: US 2007/0207498 A1
(43) Pub. Date: Sep. 6, 2007

(54) Design and construction of dimeric concanavalin a mutants

(75) Inventors: Stephen J. Palmieri, Worcester, MA (US); Dylan A. Bulseco, Princeton, MA (US); David Matzinger, Menlo Park, CA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: LifeScan, Inc., Milpitas, CA (US)

(21) Appl. No.: 11/513,003

(22) Filed: Aug. 30, 2006

Related U.S. Application Data
(63) Continuation-in-part of application No. 11/363,373, filed on Feb. 24, 2006.
(60) Provisional application No. 60/655,756, filed on Feb. 24, 2005.

(51) Int. Cl. G01N 33/53; C07K 14/42

(52) U.S. Cl. 435/7.1; 530/370

(57) ABSTRACT

Embodiments of the invention provide for compositions comprising purified polypeptides such as purified Concanavalin A (ConA) mutants. In addition, embodiments provide for polypeptides and nucleic acids encoding those polypeptides, such as mutant ConA with reduced dimer-dimer interactions compared to wild type ConA. Some embodiments also provide for sensors comprising the polypeptides disclosed herein. The embodiments also provide an improved method of producing recombinant mutant ConA.

### CROSS REFERENCE TO RELATED APPLICATION

This application is a continuation-in-part of U.S. application Ser. No. 11/363,373 entitled “Methods of Expressing, Purifying and Characterizing Concanavalin A, Mutants Thereof, and Sensors Including the Same” filed Feb. 24, 2006, which claims priority to U.S. Provisional Patent Application Ser. No. 60/655,756 filed on Feb. 24, 2005,which are herein incorporated by reference in their entirety.

### FIELD OF THE INVENTION

This invention relates to dimeric mutant Concanavalin A constructs and methods of expressing, purifying and characterizing the constructs. The invention also includes sensors incorporating purified Concanavalin A mutants.

### BACKGROUND OF THE INVENTION

Lectins are a family of carbohydrate binding proteins found both in prokaryotes and eukaryotes including: classical lectins, which are plant derived; and carbohydrate binding proteins derived from animals. Studies have identified the structure of lectin genes in soybeans, French beans and peas, as well as other sources. Concanavalin A (ConA), refers to a family of tetrameric plant lectins composed of four 26 kDa monomeric subunits that recognize and bind to carbohydrates. Purified ConA from jack beans (Canavalia ensiformis) is commonly used as a molecular probe for the investigation of glycoproteins. It specifically binds D-mannose and D-glucose with high affinity, and also binds proteins independent of glycosylation state.

ConA is initially synthesized as a precursor protein (pre-pro ConA) that undergoes multiple post-translational modifications required for activation (Sheldon, P. S. et al., Biochem J, 1996. 320 (Pt 3): 865-70; Carrington, D. M., A. Auffret, and D. E. Hanke, Nature, 1985. 313(5997): 64-7). In the plant, these modifications include removal of the signal peptide, deglycosylation, proteolytic cleavage, transposition and re-ligation (transpeptidation) of the N- and C-terminal halves to generate the mature 26 kDa ConA monomer. Monomeric ConA assembles into tetramers through a dimer intermediate in a pH dependent manner. Analyses of commercially available sources of ConA purified from jack bean meal reveal the presence of other contaminating protein bands (e.g., 14 kDa and 12 kDa) as determined by SDS-PAGE, presumably resulting from incomplete ligation of the processed peptide fragments. The incompletely processed fragments are still capable of assembling into functional tetramers with other fragments or with full-length monomers. As a result, purified commercial natural ConA tetramers include both full length and fragmented ConA monomers.

ConA's ability to specifically bind D-mannose and D-glucose with high-affinity makes it useful as a tool for determining the blood and tissue glucose levels in patients with diabetes. In particular, ConA can be useful in the design and manufacture of devices for the measurement of glucose in biological fluids, particularly blood.

However, currently available ConA tetramers are difficult to produce in commercial quantities, with sufficient purity and with the consistency desired for either a human diagnostic product or a reliable research tool. Accordingly, there exists a need in the art for the construction of stable ConA mutants with increased solubility and reduced valency.

### SUMMARY OF THE INVENTION

Embodiments of the invention provide for compositions comprising purified polypeptides such as purified Concanavalin A (ConA) mutants. In addition, embodiments provide for polypeptides and nucleic acids encoding those polypeptides, such as mutant ConA with reduced dimer-dimer interactions compared to wild type ConA. Some embodiments also provide for sensors comprising the polypeptides disclosed herein. The embodiments also provide an improved method of producing recombinant mutant ConA.

In one aspect, an exemplary embodiment is directed to a purified mutant Concanavalin A (ConA) protein including the amino acid sequence of SEQ ID NO: 16. The sequence can include a substitution at amino acid residue 58, and a substitution at one or more of amino acid residue 1I18, amino acid residue 121, and amino acid residue 192. The purified mutant Con A can have reduced dimer-dimer affinity compared to a corresponding wild type ConA protein. Purified mutant ConA proteins can include at least two, three, or four substitutions.

In some embodiments, an amino acid residue selected from the group consisting of asparagine, cysteine, proline, glutamine, tyrosine, and glycine is substituted for an amino acid residue at one or more of positions 58, 118, 121, and 192 of SEQ ID NO: 16. In an exemplary embodiment, an asparagine is substituted for the aspartic acid residue at position 58, a cysteine is substituted for the asparagine residue at position 118, a cysteine is substituted for the histidine residue at position 121, and/or a glutamine is substituted for the glutamic acid residue at position 192 of SEQ ID NO: 16. In other embodiments, at least one of the substitutions replaces a naturally occurring amino acid residue with cysteine. The purified mutant ConA protein can be substantially a dimer.

The purified mutant ConA protein can be at least about 95% pure. In exemplary embodiments, the purified mutant ConA protein is at least about 97% pure. The purified mutant ConA protein can be greater than about 95% by weight of the total protein of the composition. In some embodiments, the purified mutant ConA protein can have a purity greater than about 95% as determined by relative peak area integration, or preferably a purity greater than about 97% as determined by relative peak area integration. The purified mutant ConA can retain biological activity, such as carbohydrate binding.

In some embodiments, the purified mutant ConA protein can also include a label. The label can be a detectable label such as, for example, a radioactive label (e.g., a radioisotope), a fluorescent label, an enzyme (e.g., an enzyme, the activity of which results in a change in a detectable signal such as a change in color or emission, for instance fluorescence), a proximity-based signal generating label (e.g., a FRET component), a homogeneous time resolved fluorescence (HTRF) component, a luminescent oxygen channeling assay (LOCI) component, biotin, avidin, or another functionally similar substance, an antibody (e.g., a primary or a secondary antibody), or a portion thereof (e.g., an antigen binding portion of an antibody).

In another aspect, an exemplary embodiment includes a device capable of sensing a change in an amount of an analyte (i.e., carbohydrate). The device includes a purified mutant ConA protein as disclosed in the present application. The sensors can include a donor, and an acceptor, with the mutant ConA protein labeled with at least one of the donor and the acceptor. In one embodiment, the sensor can include a fluorescent acceptor conjugated to a glycosylated substrate. In another embodiment, the sensor can include a fluorescent donor conjugated to a glycosylated substrate. At least a portion of the device can be implantable.

Additional aspects of the invention provide for purified, isolated nucleic acid sequences encoding mutant forms of wild-type Concanavalin A (ConA), where the mutant ConA proteins have reduced dimer-dimer affinity compared to wild-type ConA. The isolated nucleic acid sequences can include SEQ ID NO: 5, 7, 9, 11, 13, 17, 19, 21, 23 and 25 or a degenerate coding sequence, or a sequence complementary to either of these, or fragment thereof. Further embodiments encompass isolated nucleic acid sequences encoding a mutant ConA operatively linked to a promoter. A host cell that contains the nucleic acid operatively linked to a promoter and expressing the encoded protein, can also be included. Isolated nucleic acid sequences can encode mutant ConA polypeptides having the amino acid sequences set forth in SEQ ID NOS: 6, 8, 10, 12, 14, 18, 20, 22, 24, and 26, and biologically active variants thereof. Such mutant ConA polypeptides have reduced dimer-dimer affinity.

In another aspect, an exemplary embodiment is directed to a method of evaluating a carbohydrate in a sample. The sample can be contacted with a specific binding pair that can include a purified mutant ConA protein and a glycoconjugate comprising a carbohydrate moiety. The purified mutant ConA and glycoconjugate can reversibly bind to each other. The extent to which carbohydrate present in the sample displaces glycoconjugate bound to the purified mutant ConA, and reversibly binds to the purified mutant ConA, can be determined subsequently. At least one of the purified mutant ConA protein and the glycoconjugate can have a detectable label.

The methods can be carried out with a sample obtained from the body of a subject (e.g., it can be a sample of urine, blood; plasma, or saliva, homogenized cells, a cell extract or an intracellular, extracellular or interstitial fluid). The sample can also be a cellular homogenate or extract. The carbohydrate of interest within such samples (i.e., the analyte) can be a monosaccharide, a disaccharide, a polysaccharide, glucose, a carbohydrate that is a component of another molecule or a supramolecular structure (e.g., a macromolecule), or combination thereof. For example, the analyte can be the carbohydrate moiety of a glycoprotein. The glycoconjugate can include, but is not limited to, one or more glycosylated serum albumin molecules, preferably of human or bovine origin, that are capable of binding to a purified mutant ConA with reduced dimer-dimer affinity. Such glycoconjugates can be useful in methods carried out in vivo or ex vivo.

### BRIEF DESCRIPTION OF THE DRAWINGS

FIG. 1 is a table of ConA mutants constructed and purified, indicating the mutations and the quarternary structure of the purified polypeptide (tetramer (T), dimer (D), or mixed tetramer/dimer (M));

FIG. 2 shows a full alignment of six Canavalia sp. (ensiformis, brasiliensis, gladiata, virosa, maritima and lineata) using CLUSTAL W (1.83);

FIG. 3 shows an alignment of differing amino acids at positions 21, 70, 129, 151, 155, 168, 202, and 208 between six Canavalia sp. (ensiformis, brasiliensis, gladiata, virosa, maritima and lineata) and two modifications of Canavalia ensiformis (mConA and the stable dimer pET32);

FIG. 4 shows an alignment comparing the dimer mutant ConA (pET32) with other Canavalia sp. at the substitution positions (amino acids 58, 118, 121, and 192);

FIG. 5 is a depiction of the structure of ConA when mutations are introduced at positions 58, 118, 121, and 192 showing a stable mutant ConA dimer with mutations D58N, N118C, H121C, and E192Q;

FIG. 6 is a graphical depiction of the SEC-MALS (size-exclusion chromatography equipped with multiangle light scattering) characterization showing that pET32, the quad mutant ConA (D58N, N118C, H121C, and E192Q), is a stable dimer of high purity (∥98%);

FIG. 7 is a graphical depiction of the SEC-MALS characterization showing that the quint mutant ConA, pET32F, (D58N, N118C, H121C, L142F and E192Q) is a stable dimer of high purity (˜98%);

FIG. 8 is a representative graphical depiction of the SEC-MALS characterization of the ConA mutants (pET26, pET29, pET31, pET33) showing that pET26, a triple mutant ConA (G58N, N118C, E192Q), forms a stable dimer, but purifies as a mixture of dimer/tetramer with approximately 50-80% dimer;

FIG. 9 shows an alignment of ConA residues for glucose binding and/or metal coordination (residues 14, 99, 100, 208, and 228);

FIG. 10 is a fluorescence emission spectra showing the FRET response upon the addition of glucose to the purified dimer mutant ConA labeled with Cy3.5b, combined with Alexa-labeled Human Serum Albumin (HSA), where the boxes show the FRET spectra before addition of glucose, and the circles show the response to glucose addition;

FIG. 11 is a time-based ratio scan of the ratio of the fluorescence intensities at 600 and 700 nm for the purified dimer mutant ConA, labeled with Cy3.5b (donor) combined with Alexa-labeled HSA (acceptor);

FIG. 12 is a graph of the results of a competition binding assay, showing that the affinity of dimer ConA mutant (Kᵢ˜21 nM) is lower than a ConA tetramer (Kᵢ˜9.1 nM) by approximately two-fold;

FIG. 13 is a fluorescence emission spectra showing the ˜262% FRET response to the addition of 500 mg/dL glucose to sensors made with Cy3.5-labeled pET32 dimer mutant ConA (donor) and Alexa647-labeled superoxide dismutase (SOD) (acceptor) at a ratio of 6 μM/24 μM; and

FIG. 14 is a fluorescence emission spectra showing the ˜266% FRET response to the addition of 500 mg/dL glucose to sensors made with Cy3.5-labeled pET32 dimer mutant ConA (donor) and Cy5.5-labeled superoxide dismutase (SOD) (acceptor).

### DETAILED DESCRIPTION OF THE INVENTION

### Definitions

Various terms relating to the biological molecules of the present invention are used throughout the specification and claims.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein.

“Nucleotide sequence” or “polynucleotide,” as used interchangeably herein refers to any polyribonucleotide or polydeoxyribonucleotide of at least 180 nucleotides in length. “Polynucleotides” include, without limitation, single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. For example, in some embodiments, the invention provides isolated nucleic acids that encode mutant ConA proteins with reduced dimer-dimer affinity when compared to wild-type ConA. The nucleic acids can include: (A) contiguous nucleotides 193-290 of SEQ ID NOS: 5, 7, 9, 11, and 13, or nucleotides 172-269 of SEQ ID NOS: 17, 19, 21, 23, and 25 such as, but not limited to, plus strand RNAs (e.g., mRNAs) and cDNAs; or (B) a nucleotide sequence complementary to contiguous nucleotides 193-290 of SEQ ID NOS: 5, 7, 9, 11, and 13, or nucleotides 172-269 of SEQ ID NOS: 17, 19, 21, 23, and 25, such as, but not limited to, minus strand RNAs (e.g., genomic or cloned RNAs) and cDNAs; or (C) fragments of (A) or (B), such fragments being at least about 180 nucleotides long beginning from about position 193 of SEQ ID NOS: 5, 7, 9, 11, and 13, or from about position 172 of SEQ ID NOS: 17, 19, 21, 23, and 25. Nucleic acid positions 193-195 of SEQ ID NOS: 5, 7, 9, 11, and 13 encode amino acid 58, which has been mutated as described in the present application. In some exemplary embodiments, the fragment spans the glucose binding site, or are at least about 642 nucleotides long, encoding for amino acid residues 14 to 228 of SEQ ID NOS: 16, 18, 20, 22, 24, or 26. It is understood by the skilled artisan that embodiments of the present invention encompass nucleic acids, i.e., RNAs, in which uracil residues (“U”) replace the thymine residues (“T”) (e.g., in SEQ ID NOS: 5, 7, 9, 11, 13, 15, 17, 19, 21, 23, and 25).

The term polynucleotide also includes DNAs or RNAs containing one or more modified bases and DNAs or RNAs with backbones modified for stability or for other reasons. “Modified” bases include, for example, locked nucleic acids (LNAs), tritylated bases and unusual bases such as inosine. A variety of modifications can been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

The term “protein” refers to a polymer of amino acids of any length, i.e., a polypeptide, and does not refer to a specific length of the product; thus, “polypeptides”, “peptides”, and “oligopeptides”, are included within the definition of “protein”, and such terms are used interchangeably herein with “protein”. The term “protein” also includes post-expression modifications of the polypeptide, for example, glycosylations, acetylations, phosphorylations and the like. Included within the definition of “protein” are, for example, polypeptides containing one or more analogs of an amino acid (including, for example, unnatural amino acids, etc.), polypeptides with substituted linkages, as well as other modifications known in the art, both naturally occurring and non-naturally occurring. Methods of inserting analogs of amino acids into a peptide sequence are known in the art. A mutant ConA protein refers to a chain of amino acids of any length, regardless of post-translational modifications, as long as the protein is biologically active (e.g., can bind a glycoconjugate).

“Variant” as the term is used herein, is a protein that differs from a reference protein (i.e. a mutant ConA protein consistent with embodiments of the present invention), but retains essential properties (i.e., biological activity), and at least one substitution at amino acid residue 58, amino acid residue 118, amino acid residue 121, and amino acid residue 192, wherein the substituted amino acid residue is replaced with a non-native amino acid at that position. In some examples, the substituted amino acid residue is selected from the group of asparagine, cysteine, proline, glutamine, serine, tyrosine, and glycine. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. Generally, differences are limited so that the sequences of the reference polypeptide and the variant are closely similar overall and, in many regions, identical.

A variant and reference protein may differ in amino acid sequence by one or more substitutions, additions, and deletions in any combination. A substituted or inserted amino acid residue may or may not be one encoded by the genetic code. A variant of a protein may be naturally occurring such as an allelic variant, or it may be a variant that is not known to occur naturally. Non-naturally occurring variants of polynucleotides and polypeptides may be made by mutagenesis techniques or by direct synthesis. For instance, a conservative amino acid substitution may be made with respect to the amino acid sequence encoding the polypeptide.

Variant proteins encompassed by the present application are biologically active, that is they continue to possess the desired biological activity of the native protein, as described herein. The term “variant” includes any polypeptide having an amino acid residue sequence substantially identical to a sequence specifically shown herein in which one or more residues have been conservatively substituted with a functionally similar residue, and which displays the ability to mimic the biological activity of a mutant ConA protein, such as for example, reduced dimer-dimer affinity when compared to wild-type ConA and/or binding to glycoconjugates. “Biological activity,” as used herein refers to the ability of the protein to bind glycoconjugates, as can be tested by methods known to one skilled in the art, such as, but not limited to, BIAcore or isothermal titration calorimetry (ITC) using glucose as the ligand. Variants may result from, for example, genetic polymorphism or from human manipulation. Biologically active variants of a mutant ConA protein of the invention will have at least about 80%, 85%, 90%, 91%, 92%, 93%, 94%, 95%, 96%, 97%, 98%, 99% or more sequence identity to the amino acid sequence for the mutant ConA protein as determined by sequence alignment programs and parameters described elsewhere herein. A biologically active variant of a protein consistent with an embodiment of the invention may differ from that protein by as few as 1-15 amino acid residues, as few as 1-10, such as 6-10, as few as 5, as few as 4, 3, 2, or even 1 amino acid residue.

The term “mutant”, as used herein, refers to an amino acid sequence that is altered by one or more amino acids. The mutant can have “conservative” changes, wherein a substituted amino acid has similar structural or chemical properties, “non-conservative” changes, or “silent” changes, or a combination thereof. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In some embodiments, a mutant can have “nonconservative” changes, e.g., replacement of a leucine with a methionine. The term mutant is also intended to include minor variations such as amino acid deletions or insertions, or both, that do not disrupt the biological activity (i.e., glycoconjugate binding) of the protein.

The term “substitution”, as used herein, refers to the replacement of one or more amino acids or nucleotides by different amino acids or nucleotides, respectively. The term “substitution” also includes the use of a chemically derivatized residue in place of a non-derivatized residue, provided that such polypeptide displays the requisite biological activity.

“Chemical derivative” refers to a subject polypeptide having one or more residues chemically derivatized by reaction of a functional side group. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and omithine may be substituted for lysine. The polypeptide also includes any polypeptide having one or more additions and/or deletions of residues, relative to the sequence of an inventive polypeptide whose sequence is shown herein, so long as the requisite biological activity is maintained.

The term “substantially the same” when referring to nucleic acid or amino acid sequences, refers to nucleic acid or amino acid sequences having sequence variations that do not materially affect the nature of the protein (i.e., the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

Some embodiments of the present invention encompass a polypeptide having substantially the same amino acid sequence set forth in SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24 or SEQ ID NO: 26. As employed herein, the term “substantially the same amino acid sequence” refers to amino acid sequences having at least about 80%, still more preferably about 90% amino acid identity with respect to a reference amino acid sequence; with greater than about 95% amino acid sequence identity being especially preferred. A “substantially the same amino acid sequence” encodes for a mutant ConA protein that retains biological activity, and reduced dimer-dimer affinity. It is recognized, however, that polypeptide containing less than the described levels of sequence identity arising as splice variants or that are modified by conservative amino acid substitutions are also encompassed within the scope of the present invention. The degree of sequence homology is determined by conducting an amino acid sequence similarity search of a protein data base, such as the database of the National Center for Biotechnology Information (NCBI), using a computerized algorithm, such as PowerBLAST, QBLAST, PSI-BLAST, PHI-BLAST, gapped or ungapped BLAST, or the “Align” program through the Baylor College of Medicine server. (E.g., Altchul, S. F., et al., Gapped BLAST and PSI-BLAST: a new generation of protein database search programs, Nucleic Acids Res. 25(17):3389-402 [1997]; Zhang, J., & Madden, T. L., PowerBLAST: a new network BLAST application for interactive or automated sequence analysis and annotation, Genome Res. 7(6):649-56 [1997]; Madden, T. L., et al., Applications of network BLAST server, Methods Enzymol. 266:131-41 [1996]; Altschul, S. F., et al., Basic local alignment search tool, J. Mol. Biol. 215(3):403-10 [1990]). Preferably, an NCBI BLAST program can be used to determine the degree of sequence homology between the sequences.

With respect to single-stranded nucleic acid molecules, the term “specifically hybridizing” refers to the association between two single-stranded nucleic acid molecules of sufficient complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

With respect to oligonucleotide constructs, but not limited thereto, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide construct with a substantially complementary sequence contained within a single-stranded DNA or RNA molecule consistent with an embodiment of the invention, to the substantial exclusion of hybridization of the oligonucleotide with single-stranded nucleic acids of non-complementary sequence.

A “coding sequence” or “coding region” refers to a nucleic acid molecule having sequence information necessary to produce a gene product, when the sequence is expressed.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g., enhancers and regulators) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “nucleic acid construct” or “DNA construct” is sometimes used to refer to a coding sequence or sequences operably linked to appropriate regulatory sequences and inserted into a vector for transforming a cell, in vitro or in vivo. This term may be used interchangeably with the term “transforming DNA”. Such a nucleic acid construct may contain a coding sequence for a gene product of interest, along with a selectable marker gene and/or a reporter gene.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The term “DNA construct”, as defined above, is also used to refer to a heterologous region, particularly one constructed for use in transformation of a cell. A cell has been “transformed” or “transfected” or “transduced” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

As used herein, the terms “recombinant polynucleotide” and “polynucleotide construct” are used interchangeably to refer to linear or circular, purified or isolated polynucleotides that have been artificially designed, and which comprise at least two nucleotide sequences that are not found as contiguous nucleotide sequences in their initial natural environment.

The term “recombinant polypeptide” is used herein to refer to polypeptides that have been artificially designed, and which comprise at least two polypeptide sequences that are not found as contiguous polypeptide sequences in their initial natural environment, or to refer to polypeptides which have been expressed from a recombinant polynucleotide.

As used herein, the terms “vector” and “vehicle” are used interchangeably in reference to nucleic acid molecules that transfer DNA segment(s) from one cell to another.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals.

The term “mConA” refers to a mutant Concanavalin A comprising the nucleic acid and polypeptide sequence of SEQ ID NO: 1 and 2, respectively, containing a D58G mutation which converts this region of ConA from C. ensiformis (amino acids VDKRL) into the sequence found in C. gladiata (amino acids VGKRL).

The term “wild type ConA” refers to either the nucleic acid sequence or the polypeptide sequence of any mature form of native Concanavalin A. In some embodiments, it refers to recombinant Concanavalin A derived from C. ensiformis or C. gladiata, the polypeptide sequences of which are shown in SEQ ID NO: 3 and SEQ ID NO: 4 respectively. The term “gConA” refers to recombinant wild type Concanavalin A comprising the polypeptide sequence of SEQ ID NO:4 derived from C. gladiata.

As used herein, the term “substantially a dimer” is intended to mean that the purified protein is at least 50% dimer, preferably about 60% dimer, more preferably 70%, 80%, 90%, 95%, 96%, 97% or 98%. The percent dimer can be measured by a number of methods known in the art. For example, the percent dimer of the purified mutant ConA can be determined using SEC-MALS (size-exclusion chromatography equipped with multiangle light scattering).

The term “glycoconjugate”, as used herein, refers to a conjugate that binds specifically and reversibly to a mutant ConA consistent with embodiments of the present invention. A glycoconjugate includes a carbohydrate, a label moiety, and preferably, a carrier molecule. Non-limiting examples of suitable carbohydrates include glucose, fructose, sucrose, mannose, monosaccharides, and oligosaccharides. The carbohydrate should be the same as the analyte carbohydrate to be detected in a sample. The analyte carbohydrate should competitively inhibit binding of the glycoconjugate to the mutant ConA. The label can be, for example, a FRET component, a HTRF component, a LOCI component or other functionally similar substances.

In FRET-based applications the label is a FRET component. In some embodiments, the carbohydrate and the FRET component are both bound to a carrier molecule. The carrier molecule is nonreactive with substances found in the sample, provides a site at which a carbohydrate can be bound, and provides a site at which a FRET component can be bound. The carrier molecule should not interfere with the binding between the conjugated carbohydrate and the reduced valency mutant Con A. Suitable carriers include proteins, such as bovine, or human serum albumin, β-lactoglobulin, superoxide dismutase (SOD), immunoglobulins, antibodies, glycoproteins or glycolipids containing the carbohydrate moiety recognized by the mutant ConA protein, and synthetic polymers to which the carbohydrate is covalently coupled. Methods of coupling FRET components to carrier molecules are known to those skilled in the art and incorporated herein by reference (Hermanson, 1996, Bioconjugate Techniques, Academic Press, Inc).

A FRET component can be either a donor or an acceptor of energy. If the energy absorbing FRET donor is coupled to the glycoconjugate, then the energy absorbing FRET acceptor is coupled to the mutant ConA. If the energy absorbing FRET acceptor is coupled to the glycoconjugate, then the energy absorbing FRET donor is coupled to the mutant ConA.

The term “implantable” refers to a device that is intended for both short-term (i.e., a few days but less than one month) and long-term implantation within the body of a subject, (i.e., implantation for periods of one month or longer). Implantable devices can be placed either subcutaneously or in a blood vessel. As used herein, the term implantable also refers to percutaneous devices. For example, implantable percutaneous sensors can be needlelike or can be inserted through a needle and are designed to operate for a few days and be replaced by the subject.

The term “subject” as used herein refers to any living organism capable of eliciting an immune response. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, are intended to be covered.

Certain exemplary embodiments will now be described to provide an overall understanding of the principles of the structure, function, manufacture, and use of the compositions and methods disclosed herein. One or more features of these embodiments are illustrated in the accompanying figures. Those of ordinary skill in the art will understand that the compositions and methods specifically described herein and illustrated in the accompanying figures are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Polypeptides and Nucleic Acids

The scope of the present invention includes both polypeptides and nucleic acids encoding said polypeptides.

I. Nucleic Acids

In one aspect, the invention relates to isolated nucleic acids that encode mutant ConA proteins with reduced dimer-dimer affinity compared to wild-type ConA. The reduction in dimer-dimer affinity can be shown by any method known in the art for determining oligomeric structure, including, but not limited to, SEC-MALS, comparison of amount of tetramer versus dimer purified from an affinity column, sedimentation analysis using an analytical ultracentrifuge, native electrophoresis, electron microscopy and X-ray crystallography.

SEQ ID NOS: 5, 7, 9, 11, 13, 17, 19, 21, 23, and 25 are mutant nucleic acid sequences of ConA, encoding for mutant ConA polypeptides with the following substitution mutations:

| | |
|---|---|
| SEQ ID NOS: 5 and 17 | pET26 (D58N, N118C, E192Q) |
| SEQ ID NOS: 7 and 19 | pET 29 (D58P, N118C, E192C) |
| SEQ ID NOS: 9 and 21 | pET 31 (D58N, N118C, H121Y, E192Q) |
| SEQ ID NOS: 11 and 23 | pET 32 (D58N, N118C, H121C, E192Q) |
| SEQ ID NOS: 13 and 25 | pET 33 (D58N, N118C, H121P, E192Q) |

The sequences shown in SEQ ID NOS: 5, 7, 9, 11, 13 were engineered to include the 21 nucleic acids (atggctaccgtagcgcaagct SEQ ID NO: 27) secretion signal sequence from the E. coli outer membrane protein (ompA) at the 5′ end of the ConA coding sequence. The sequence encodes for the amino acids: MATVAQA (SEQ ID NO: 28). The nucleic acids and polypeptides consistent with embodiments of the invention are intended to include both nucleic acids and polypeptides with and without this secretion signal sequence.

The differences between mutant nucleic acid molecules and corresponding wild-type nucleic acid molecules are due to substitution of a native amino acid; and in addition, can be due to degeneracy of genetic codons. An isolated nucleic acid containing such a mutant nucleic acid sequence can be used to clone and express the mutant ConA in a host cell. A nucleic acid variant can possess the codons preferred by a particular prokaryotic or eukaryotic host. The codons may be selected to increase the rate at which expression of a polypeptide occurs in the prokaryotic or eukaryotic host in accordance with the frequency with which the codons are utilized by the host. The mutant nucleic acid can further include such variations as nucleotide substitutions, deletions, inversions, or insertions on the wild-type DNA as long as the glycoconjugate binding site of the encoded protein is preserved (as discussed below).

The above-described mutant DNA can be prepared using site-directed mutagenesis, which introduces specific nucleotide substitutions (i.e., mutations) at defined locations in a nucleic acid sequence. See, for example, Zoller and Smith (1983) Meth. Enzymol. 100: 468; and Molecular Cloning, A Laboratory Manual (1989) Sambrook, Fritsch and Maniatis, Cold Spring Harbor, N.Y., chapter 15. Alternatively, the mutant DNA may be synthesized, in whole or in part, using chemical methods well known in the art. See Caruthers et al. (1980) Nucl. Acids Res. Symp. Ser. 215 223, and Horn et al. (1980) Nucl Acids Res. Symp. Ser. 225 232. In particular, multiple mutations can be introduced through various methods based on, e.g., polymerase chain reaction (PCR), ligase chain reaction (LCR), or overlap extension polymerase chain reaction. See Ge and Rudolph (1997) BioTechniques 22: 28 30.

The mutant nucleic acid can encode a polypeptide having an amino acid sequence set forth in SEQ ID NOS: 2, 6, 8, 10, 12, 14, 18, 20, 22, 24, or 26. Alternatively, it can encode a polypeptide variant having an amino acid sequence that is 80% identical to, or differs by less than 24 amino acid residues from SEQ ID NOS: 2, 6, 8, 10, 12, 14, or 16. If alignment is needed for this comparison, the sequences can be aligned for maximum homology. The polypeptide variant is correlated with at least one biological activity of a polypeptide encoded by SEQ ID NOS: 2, 6, 8, 10, 12, 14, 16, 18, 20, 22, 24, or 26, e.g., glycoconjugate binding. A polypeptide variant may have “conservative” changes, wherein a substituted amino acid has similar structural or chemical properties. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In some embodiments, a polypeptide variant may have “nonconservative” changes, e.g., replacement of a leucine with a methionine. Further, a polypeptide variant may also include amino acid deletions or insertions, or both. Guidance in determining which amino acid residues may be substituted, inserted, or deleted without abolishing the biological activity may be found using computer programs, for example DNASTAR software, to ensure that amino acids needed for glucose binding are not disrupted.

Site-directed mutagenesis can be used to change one or more DNA residues that may result in a silent mutation, a conservative mutation, or a nonconservative mutation. Included within the scope of the invention are nucleic acid sequences that are at least about 80% identical to SEQ ID NOS: 5, 7, 9, 11, 13, 17, 19, 21, 23, or 25 over their entire length to a nucleic acid sequence encoding the polypeptide having the amino acid sequences set out herein, and nucleic acid sequences which are complementary to such nucleic acid sequences. Alternatively, highly preferred are nucleic acid sequences that comprise a region that is at least about 85% identical, more highly preferred are nucleic acid sequences that comprise a region that is at least about 90% identical, and among these preferred nucleic acid sequences, those with at least about 95% are especially preferred. Furthermore, those with at least about 97% identity are highly preferred among those with at least about 95%, and among these those with at least about 98% and at least about 99% are particularly highly preferred, with at least about 99% being the most preferred. The nucleic acid sequences which hybridize to the hereinabove described nucleic acid sequences in a preferred embodiment encode polypeptides which retain substantially the same biological activity as the polypeptide characterized by the mutant ConA amino acid sequences set forth herein. Preferred embodiments in this respect, moreover, are nucleic acid sequences that encode polypeptides that retain substantially the same biological function or activity as the mature polypeptide encoded by the DNA of SEQ ID NOS: 5, 7, 9, 11, 13, 17, 19, 21, 23, and 25. Embodiments of the present invention further relate to nucleic acid sequences that hybridize to the herein above-described sequences. In this regard, the embodiments especially relate to nucleic acid sequences that hybridize under stringent conditions to the herein above-described nucleic acid sequences. As herein used, the term “stringent conditions” means hybridization will occur only if there is at least about 95% and preferably at least about 97% identity between the sequences.

The nucleic acids may be maintained as DNA in any convenient cloning vector. Clones can be maintained, for example, in a plasmid cloning/expression vector, examples of which are included below, the plasmid being propagated in a suitable host cell.

II. Proteins and Polypeptides

Recombinant proteins and polypeptides within the scope of the present invention, may be prepared in a variety of ways, according to known methods. For example a cDNA or gene encoding for the protein of an embodiment of the invention may be cloned into an appropriate transcription vector. A host cell may be transformed with the transcription vector and the protein expressed either intracellularly or extracellularly. In some aspects of the invention, the protein is expressed intracellularly, inclusion bodies are formed, the inclusion bodies and the protein of the invention are solubilized and the protein of interest is purified from solution.

Polypeptides can contain amino acids other than the 20 amino acids commonly referred to as the 20 naturally-occurring amino acids. Further, many amino acids, including the terminal amino acids, may be modified by natural processes, such as processing and other post-translational modifications, or by chemical modification, such as fluorescent labeling, using techniques well known in the art. Common modifications that occur naturally in polypeptides are described in basic texts, detailed monographs, and the research literature, and they are well known to those of skilled in the art.

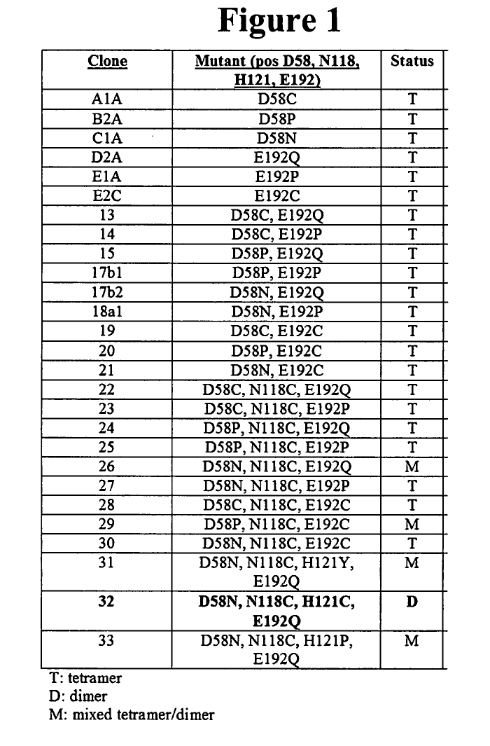

Wild-type ConA purified from natural sources does not consist of identical subunits. While the monomers of wild-type ConA purified from natural sources are structurally identical, the primary structure may either be contiguous or fragmented (14kd and 11kd fragments) due to incomplete transpeptidation. The monomeric subunits of both wild-type ConA purified from natural sources and wild-type ConA produced recombinantly associate into tetramers at physiological pH. Each monomeric subunit is approximately 27 kDa in mass and contains one carbohydrate binding site. Accordingly, tetrameric ConA is capable of binding four carbohydrate molecules. The number of carbohydrate binding sites also can be referred to as valency. Thus, tetrameric ConA has a valency of four. The mutant ConA proteins of the present invention are composed of mutated monomeric subunits that associate into dimers at physiological pH (See, FIG. 1).

Three-dimensional crystallographic studies of ConA have demonstrated that in dimeric ConA, one monomeric subunit is paired across a two fold axis of symmetry with the second monomeric subunit, and that these dimers in turn are paired across 222 (D₂) points of symmetry to form tetramers (Becker et al., (1975), J. Biol. Chem. 250:1513-1524; Reeke et al., J. Biol. Chem. (1975), 250:1525-1546). Although crystal structure information suggests that certain amino acids may play a role in dimer-dimer association, the specific combination of residues that needed to be mutated and the identity of the mutations were not obvious in light of the information. As shown in FIG. 1, at least three amino acids had to be mutated to specific amino acids in order to produce a polypeptide with reduced dimer-dimer affinity compared to wild-type ConA, that is stable, soluble, and retains a biological activity (i.e., the ability to bind glycoconjugates) of wild-type ConA. While reduced valency ConA dimers have been produced through chemical modification (i.e., succinylation of tetrameric ConA), reduced valency ConA has not been produced through recombinant methods prior to this invention. Furthermore, none of the previous studies addressed large-scale purification of exogenously expressed ConA, or even appreciated the in vitro application issues with tetrameric ConA, such as the difficulty in purification. The use of the lower valency ConA mutants of the present invention reduces protein precipitation in the presence of a bacterial host contaminant.

As shown in FIG. 1, a variety of mutants were constructed and purified and tested for quarternary structure (tetramer (T), dimer (D), or mixed tetramer/dimer (M)). The following exemplary sequences produced stable proteins with reduced dimer-dimer affinity:

| | |
|---|---|
| SEQ ID NOS: 6 and 18 | pET26 (D58N, N118C, E192Q) |
| SEQ ID NOS: 8 and 20 | pET 29 (D58P, N118C, E192C) |
| SEQ ID NOS: 10 and 22 | pET 31 (D58N, N118C, H121Y, E192Q) |
| SEQ ID NOS: 12 and 24 | pET 32 (D58N, N118C, H121C, E192Q) |
| SEQ ID NOS: 14 and 26 | pET 33 (D58N, N118C, H121P, E192Q) |

The sequences shown in SEQ ID NOS: 6, 8, 10, 12, and 14 were engineered to include the seven amino acids secretion signal sequence from the E. coli outer membrane protein (ompA).

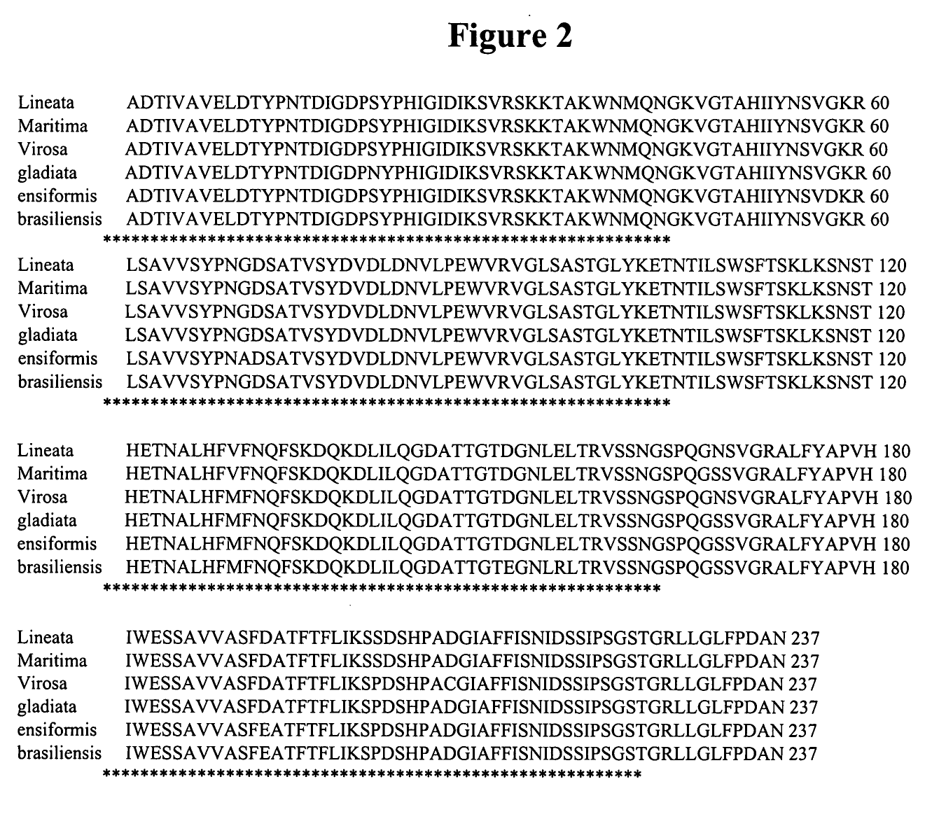

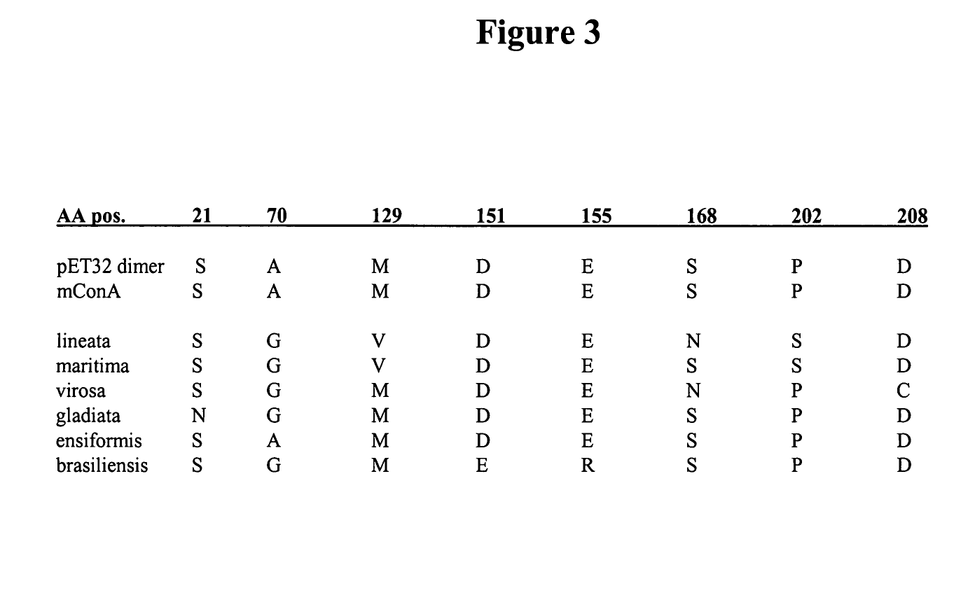

As shown in the alignment of the amino acid sequence of the ConA polypeptide from six Canavalia sp. (ensiformis, brasiliensis, gladiata, virosa, maritima and lineata), which have the same tertiary and quaternary structure, using CLUSTAL W (1.83), the sequence is well conserved and differs only at eight positions, specifically amino acids at positions 21, 70, 129, 151, 155, 168, 202, and 208 (See, FIGS. 2 and 3). Accordingly, as shown in SEQ ID NO: 16, these eight amino acid positions can be substituted without affecting the biological activity of the protein. In some embodiments, the purified mutant ConA protein comprises the amino acid sequence of SEQ ID NO: 16 with a substitution at amino acid residue 58 and a substitution of at least one of amino acid residue 118, amino acid residue 121, and amino acid residue 192. Positions 21, 70, 129, 151, 155, 168, 202, and 208 of SEQ ID NO: 16 can be any amino acid residue. In an exemplary embodiment, position 21 of SEQ ID NO: 16 is selected from the group consisting of serine and asparagine; position 70 of SEQ ID NO: 16 is selected from the group consisting of alanine and glycine; position 129 of SEQ ID NO: 16 is selected from the group consisting of methionine and valine; position 151 of SEQ ID NO: 16 is selected from the group consisting of aspartic acid and glutamic acid; position 155 of SEQ ID NO: 16 is selected from the group consisting of glutamic acid and arginine; position 168 of SEQ ID NO: 16 is selected from the group consisting of serine and asparagine; position 202 of SEQ ID NO: 16 is selected from the group consisting of serine and proline; position 208 of SEQ ID NO: 16 is selected from the group consisting of aspartic acid and cysteine.

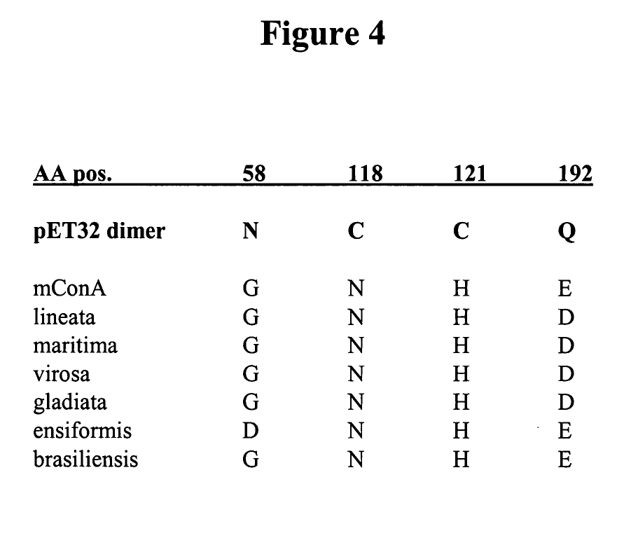

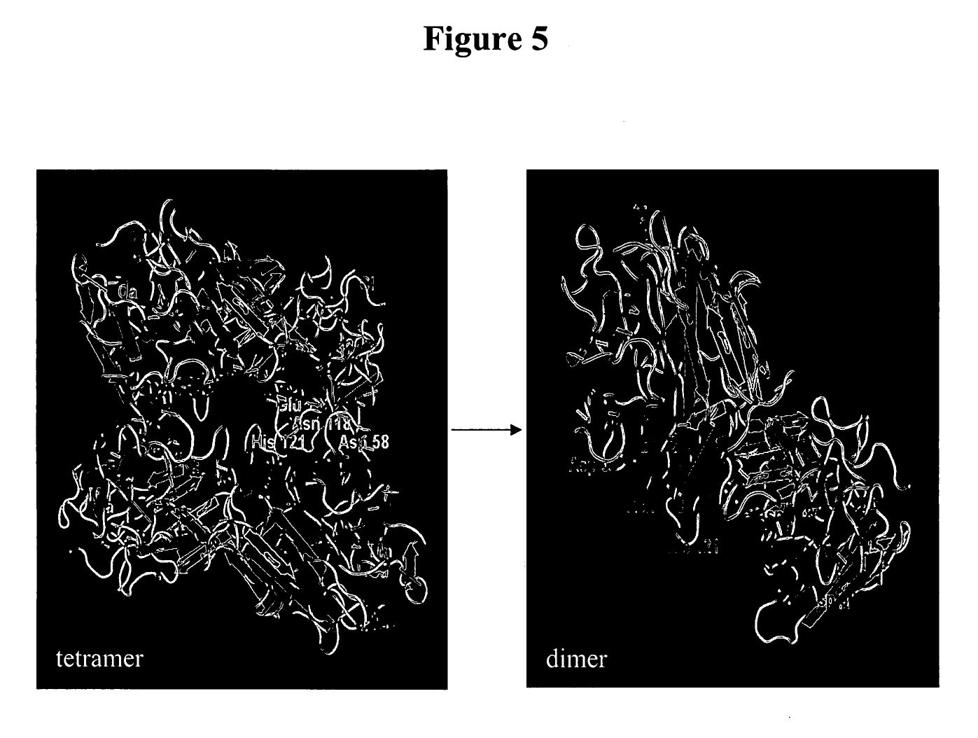

FIG. 4 shows an alignment comparing the dimer mutant ConA (pET32) with other Canavalia sp. at the substitution positions (amino acids 58, 118, 121, and 192). The mutations at amino acids 58, 118, 121, and 192 are capable of disrupting the dimer-dimer interactions. All four of these amino acids contribute to a number of bonding interactions between the dimers, such as protein-protein H-bonds, H-bonds via water, and Van der Walls contacts. These four amino acids contribute approximately 64.5% of the total number of interactions necessary for tetramerization. FIG. 5 is a depiction of the structure of ConA when mutations are introduced at positions 58, 118, 121, and 192. The structural depiction shows that a stable mutant ConA dimer is produced with mutations D58N, N118C, H121C, and E192Q.

Polypeptides within the scope of the present invention include a polypeptide having the amino acid sequence set forth in SEQ ID NOS: 2, 6, 8, 10, 12, 14, 16, 18, 20, 22, 24, or 26 (in particular, the mature polypeptide, e.g., residues 1 to 235 of SEQ ID NO: 24) as well as polypeptides which have at least about 80% identity (e.g., at least about 90%, 95%, or 99% identity) to the amino acid sequence set forth in SEQ ID NOS: 2, 6, 8, 10, 12, 14, 16, 18, 20, 22, 24, or 26. Polypeptides of the invention also include fragments of the amino acid sequence set forth in SEQ ID NOS: 2, 6, 8, 10, 12, 14, or 16, or fragments having at least about 80% sequence identity to the amino acid sequence set forth in SEQ ID NOS:2, 6, 8, 10, 12, 14, 16, 18, 20, 22, 24, or 26; where such fragments are at least 60 amino acids in length and span at least two of amino acid residues at wild-type positions 58, 118, 121, and 192. It is important to note that SEQ ID NOS: 2, 6, 8, 10, 12, and 14 contain the seven amino acid (MATVAQA, needs sequence identifier) secretion signal sequence from the E. coli outer membrane protein (ompA) at the N-terminal end of the ConA mutant protein. Thus, wild-type positions 58, 118, 121, 192 correspond to amino acids 65, 125, 128, and 199 of SEQ ID NOS: 2, 6, 8, 10, 12, and 14. Polypeptides within the scope of this invention are intended to include polypeptides with and without this secretion signal sequence. Preferred in this aspect of the invention are fragments having structural or functional attributes of the polypeptide characterized by the sequences of SEQ ID NOS: 2, 6, 8, 10, 12, 14, 16, 18, 20, 22, 24, and 26.

An exemplary mutant of ConA (pET32) was produced wherein the amino acids at wild-type positions 58, 118, 121, and 192 were mutated as follows: D58N, N118C, H121C, E192Q. The full length mutant ConA (pET32) polypeptide sequence is depicted in SEQ ID NO: 12, and an exemplary nucleic acid sequence coding for this mutant ConA polypeptide is depicted in SEQ ID NO: 11. While these sequences contain the seven amino acid (MATVAQA, needs sequence identifier) secretion signal sequence from the E. coli outer membrane protein (ompA) at the N-terminal end of the ConA mutant protein, this sequence is not necessarily present in all embodiments of the invention. The mutations at positions 58, 118, 121, and 192 produced a highly pure (>98%) stable dimer. A mutant ConA protein that includes these four amino acid mutations can have improved ConA performance in both dye labeling and FRET reactions. In addition, such a mutant ConA protein can result in reduced precipitation during purification and conjugation to Cy dyes.

A residue that is replaced renders both the order and number of the remaining amino acids the same as the polypeptide before the residue was replaced. A residue may be replaced with a conservative or non-conservative residue. A residue that is deleted does not disturb the order of the remaining amino acids, but reduces the number of residues of the polypeptide by one. A residue that is modified is one that is chemically altered; this change does not alter the order or number of remaining amino acids in the polypeptide.

Proteins consistent with embodiments of the invention can be isolated or purified by a variety of known biochemical means, including, for example, by recombinant expression systems described herein, precipitation, gel filtration, ion-exchange, reverse-phase, and affinity chromatography, electrophoresis, and the like. Other well-known methods are described in Deutscher et al., Guide to Protein Purification: Methods in Enzymology Vol. 182, (Academic Press, [1990]).

Isolated mutant ConA proteins can also be chemically synthesized. For example, synthetic polypeptides can be produced using Applied Biosystems, Inc. Model 430A or 431 A automatic peptide synthesizer (Foster City, Calif.) employing the chemistry provided by the manufacturer and the amino acid sequences provided herein.

The mutant ConA proteins can be recombinantly produced, for example, using eukaryotic or prokaryotic cells genetically modified to express mutant ConA protein-encoding polynucleotides in accordance with the teachings described herein. Recombinant methods and expression systems are well known, as described, for example, in Sambrook et al., supra., 1989. An example of a method for preparing a mutant ConA protein is to express nucleic acids encoding the mutant ConA protein of interest in a suitable host cell that contains the expression vector and recovering the expressed polypeptide, as discussed above. A suitable host cell can include, for example, a bacterial cell, a yeast cell, an insect cell, an amphibian cell (i.e., oocyte), or a mammalian cell

“Recombinant host cells”, “host cells”, “cells”, “cell lines”, “cell cultures”, and other such terms denoting prokaryotic or eukaryotic cell lines cultured as unicellular or monolayer entities, refer to cells which can be, or have been, used as recipients for a recombinant expression vector or other foreign nucleic acids, such as DNA or RNA, and include the progeny of the original cell which has been transfected. It is understood that the progeny of a single parental cell may not necessarily be completely identical in morphology or in genomic or total DNA complement as the original parent, due to natural, accidental, or deliberate mutation.

Alternatively, a cell free system may be used for protein production. A cDNA or gene, for example, may be cloned into an appropriate in vitro transcription vector, such as pSP64 or pSP65 for in vitro transcription, followed by cell-free translation in a suitable cell-free translation system, such as wheat germ or rabbit reticulocytes. In vitro transcription and translation systems are commercially available, e.g., from Promega Biotech, Madison, Wis. or BRL, Rockville, Md.

III. Characterization of Mutant Concanavalin A (ConA)

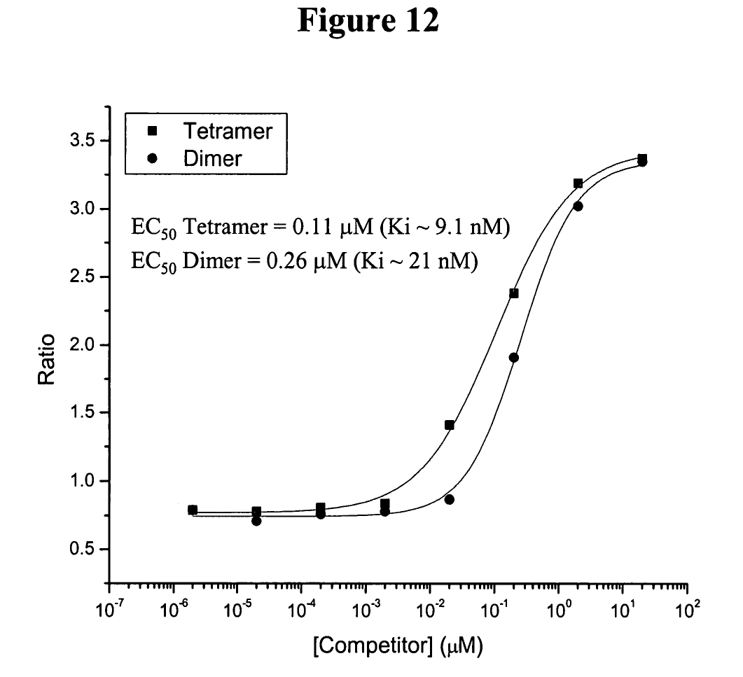

In particular, an embodiment of the invention includes polypeptides comprising one or more mutants of Concanavalin A (ConA) having reduced dimer-dimer affinity compared to a corresponding wild type ConA protein, and nucleic acids encoding such polypeptides. A particular embodiment of the invention includes mutations to the sequence encoding naturally occurring ConA that change one or more amino acids. These mutations result in a protein with improved characteristics, including reduced dimer-dimer affinity. Reduction in dimer-dimer affinity results in reduced precipitation during purification, increased solubility (i.e., less prone to aggregation), improved stability, lower toxicity due to reduced crosslinking capabilities, increased conjugation to Cy dyes, and improved brightness. Lower affinity glucose binding protein would require higher concentrations to achieve optimal dynamic range, which would result in higher brightness. Since dimeric mutant ConA has slightly lower affinity for glucose binding (EC₅₀ Tetramer=0.11 μM (Kᵢ˜9.1 nM), EC₅₀ Dimer =0.26 μM (Kᵢ˜21 nM); See FIG. 12), the use of dimeric mutant ConA polypeptides consistent with embodiments of the present invention allows for higher concentrations and therefore greater brightness. In addition, improved brightness can also be achieved with the dimeric mutant ConA constructs described herein since they can be labeled to higher dye levels without detrimental effects on glucose binding.

Since ConA polypeptides are subunits of a multimeric molecule, i.e., a tetramer formed from two associated dimers, mutations in a ConA polypeptide can alter the ability of the ConA polypeptide to assemble into tetramers. For example, ConA polypeptide can be modified such that subunits do not assemble into tetramers, but rather are present as monomers, dimers, or trimers. The nucleic acid encoding the ConA polypeptide can be mutagenized at residues important in monomer-monomer interactions to produce a monomer which does not assemble into dimers, or tetramers. For example, one or more of amino acid positions 58, 118, 121, and 192 can be mutagenized. The nucleic acid encoding the ConA polypeptide also can be mutagenized at residues important in dimer-dimer interactions to produce dimers which do not assemble into tetramers.

In addition, the mutant ConA polypeptides can have reduced valency, which results in simpler binding relationships, and therefore a simpler overall sensor system. Mutant ConA polypeptides having reduced valency refers to ligands which have been genetically engineered to have less than the normal valency, i.e., a valency less than 4. Thus, mutant ConA polypeptides consistent with an embodiment of the present invention can be designed to have as few carbohydrate binding sites as desired, preferably three or fewer and, preferably, a single carbohydrate binding site. For example, the reduced valency mutant ConA polypeptides can have a single carbohydrate binding site and be a monomeric molecule, e.g., a monomeric mutant ConA polypeptide. The mutant ConA polypeptides can have at least one and preferably two fewer carbohydrate binding sites than the naturally occurring multimeric molecule.

Mutant ConA polypeptides consistent with an embodiment of the present invention can be one member of the specific binding pair and can interact with the carbohydrate coupled to the glycoconjugate, the second member of the specific binding pair. The reduced mutant ConA polypeptides can be coupled to a proximity based signal generating label moiety (e.g., to an energy absorbing FRET component). The energy absorbing FRET component may either be a donor or an acceptor of energy. If the energy absorbing FRET donor is coupled to the mutant ConA polypeptides, then the energy absorbing FRET acceptor is coupled to the glycoconjugate. If the energy absorbing FRET acceptor is coupled to the mutant ConA polypeptides, then the energy absorbing FRET donor is coupled to the glycoconjugate.

Interaction between the mutant ConA polypeptides and the glycoconjugate brings the energy absorbing FRET components together permitting non-radiative energy transfer and FRET. In the presence of carbohydrate in the sample, there is competition between the glycoconjugate and the carbohydrate for binding to the mutant ConA polypeptides. As the binding site (or sites) on the mutant ConA polypeptides become occupied by carbohydrate molecules, glycoconjugate molecules are displaced or prevented from binding. This prevents the energy absorbing FRET components from moving together and failure to promote the energy transfer between the components.

a) Glycoconjugate Binding Site

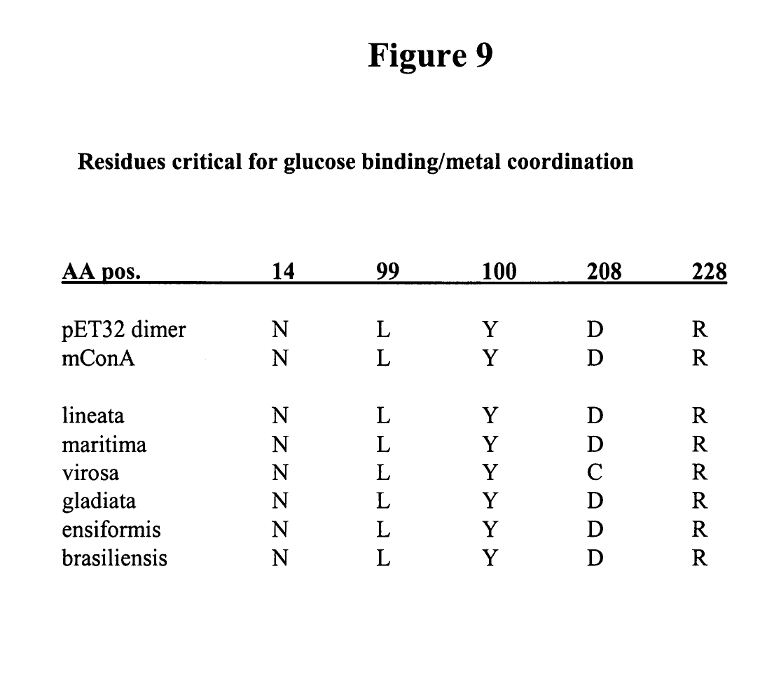

Embodiments of the invention comprise mutants of ConA that result in reduced dimer-dimer affinity while retaining its biological activity (i.e., glycoconjugate binding). ConA proteins bind glycoconjugates through a complex system involving O or N-glycosylation. For example, mannose molecules are bound to ConA in a pocket composed of two metal ions, an asparagines, aspartic acid, alanine and several water molecules (See, Ramachandraiah, G., et al. Proteins: Strucure, Function, and Genetics 39: 358-364 (2000)). FIG. 9 shows an alignment of ConA residues for glucose binding and/or metal coordination (residues 14, 99, 100, 208, and 228). Accordingly, these positions can be conserved in the construction of mutant ConA polypeptides in order to retain biological activity.

b) Production and Purification of Mutant ConA Proteins

The scope of the present invention also includes an improved process for producing and purifying mutant ConA, and in particular ConA of relatively high purity. Historically, purifying ConA from natural sources has been difficult, resulting in a number of problems. These problems include the production of a composition that contains both full length and fragmented ConA.

An exemplary embodiment is directed to a method of producing a recombinant mutant ConA by inducing expression of the mutant ConA in a bacterial cell culture that has been transformed by a vector containing a encoding the mutant ConA polypeptide of interest. The induction and presence of the ompA signal sequence ofacilitates the formation of inclusion bodies. The cells of the bacterial culture are then lysed to release an insoluble inclusion body fraction. The inclusion body fraction is then purified and the inclusion bodies are solubilized (e.g., using guanidine hydrochloride followed by sonication) so that the mutant ConA of interest is present in solution. The mutant ConA is then denatured and subsequently allowed to re-fold in solution. The solution is then purified to recover the mutant ConA of interest.

By way of example, the exemplary process can include using vectors having an antibiotic resistance gene coupled to a promoter and a nucleic acid encoding a mutant ConA polypeptide. Antibiotic resistance genes include, for example, ampicillin, kanamycin, and tetracycline.

The transformed bacterial cells can be induced either in the presence or absence of antibiotic. For example, the transformed bacterial cell culture can be induced with isopropyl β-D-thiogalactopyranoside (IPTG) in the absence of kanamycin.

Solution purification can be performed by a number of different methods, including but not limited to, affinity chromatography and size-exclusion chromatography. In one example, affinity chromatography alone is used to purify the solution. In another example, both affinity chromatography and size-exclusion chromatography are used.

The production process can be useful for producing a mutant ConA of the present application. This production and purification process results in highly purified protein, particularly highly purified recombinant protein including, e.g., mutant ConA protein having a purity of at least about 95%, at least about 96%, at least about 97%, at least about 98%, and at least about 99%.

The approximately 52 kDa purified mutant dimeric ConA protein described herein is preferably substantially free of contaminants including e.g., contaminants having molecular weights from about 10 kDa to about 20 kDa, from about 30 kDa to 40 kDa, or combinations thereof. The purification method described herein has produced ConA of sufficient purity, e.g., mutant ConA having a level of contaminants of less than about 5%, less than about 4%, less than about 3%, less than about 2%, and less than about 1%, as characterized by SEC-MALS.

IV. Sensors

Other exemplary embodiments of the invention include sensors having a purified mutant ConA as described herein. The sensors are capable of detecting the presence of an analyte. The sensors can include a reagent suitable for detecting the analyte in a liquid, e.g., body fluid such as blood or interstitial fluid. Useful reagents include, e.g., energy absorbing reagents, including light absorbing and sound absorbing reagents), x-ray reagents, spin resonance reagents, nuclear magnetic resonance reagents, and combinations thereof.

A useful class of reagents for detecting analyte includes fluorescence reagents, i.e., reagents that include a fluorophore or a compound labeled with a fluorophore. The fluorescence reagent can reversibly bind to the analyte, and the fluorescence behavior of the reagent can change when analyte binding occurs.

Changes in fluorescence associated with the presence of the analyte may be measured in several ways. These changes include changes in the excited state lifetime of, or fluorescence intensity emitted by, the fluorophore (or component labeled with the fluorophore). Such changes also include changes in the excitation or emission spectrum of the fluorophore (or component labeled with the fluorophore). Changes in the excitation or emission spectrum, in turn, may be ascertained by measuring (a) the appearance or disappearance of emission peaks, (b) the ratio of the signal observed at two or more emission wavelengths, (c) the appearance or disappearance of excitation peaks, (d) the ratio of the signal observed at two or more excitation wavelengths or (e) changes in fluorescence polarization.

The reagent can be selected to exhibit non-radiative fluorescence resonance energy transfer (FRET), which can be used to determine the occurrence and extent of binding between members of a specific binding pair.

Examples of FRET, FRET-based sensors, their use and method of manufacture, are described in U.S. Pat. Nos. 6,844,166, 6,040,194 and U.S. Publ. No. 2005-0095174, filed Oct. 31, 2003 which are hereby incorporated by reference in their entirety. Examples of other sensors are also described in U.S. Pat. Nos. 6,319,540, 6,383,767, 6,850,786, and 5,342,789, which are also hereby incorporated by reference.

The sensor can be capable of detecting the analyte based on nonradiative fluorescence resonance energy transfer. In some embodiments, the fluorescence reagent includes an energy acceptor and an energy donor. The fluorescence reagent can comprise a mutant ConA taught by the present application as a glucose binding protein and a glycosylated substrate. In some embodiments, the glycosylated substrate includes human serum albumin.

Sensors consistent with embodiments of the invention can be implantable. An implantable sensor may be provided with a selectively permeable membrane that permits the analyte (but not fluorescence reagent) to diffuse into and out of the sensor. In another embodiment, at least some of the components of the fluorescence reagent are immobilized within the sensor (e.g., on a substrate or within the pores of a porous matrix). For example, in the case of an analogue labeled with a donor and a ligand labeled with acceptor, one (or both) materials can be immobilized. In another embodiment, at least some of the components of the fluorescence reagent are freely mobile (i.e., not immobilized) within the sensor.

Specific binding pairs destined for implantation within a subject can be encapsulated (e.g., in a microcapsule). The encapsulation can substantially isolate the pair from the subject's immune system. For example, a specific binding pair can be encapsulated in a hydrogel core (e.g., an alginate or agarose core that is surrounded by an immunoisolating membrane such as a polyamino acid membrane (e.g., a polylysine membrane)). Composite microcapsules such as those described in PCT/US96/03 135 are particularly useful with the sensors and methods described herein. Other commonly used membranes for implantable biosensors include, but are not limited to, polyurethane, cellulose acetate, polypropylene, silicone rubber, and Nafion.

In some embodiments, the sensor can be used with an implantable or externally wearable infusion pump. The infusion pump may be controlled by a remote circuit via a receiver in the pump, or may be manually controlled using sensor information as a guideline. The infusion pump may be implantable or may be worn externally by the patient. These pumps can be designed with appropriate circuitry to receive and respond to output from a glucose sensor consistent with an embodiment of the present invention.

In preferred embodiments, the specific binding pair is illuminated, and the energy transfer is monitored (e.g., through the subject's skin). Energy transfer can be between the first and second energy absorbing FRET components described below. For example, one or more of the mutant ConA proteins of the present invention can be conjugated with fluorophores (such as, for example, -NHS and maleimide-based Cy3.5b and Alexa-568) and paired with a fluorescently-labeled, glycosylated protein (such as, for example, HSA) or a peptide. The paired complex can then be encapsulated within, for example, an alginate/poly-L-lysine-based bead.

Sensors consistent with embodiments of the present invention include, but are not limited to, sensors made with conjugated pairs of mutant ConA described herein and Human Serum Albumin (“HSA”); conjugated pairs of mutant ConA and superoxide dismutase (SOD); and conjugated pairs of mutant ConA and BSA. Either the mutant ConA or the glycoconjugate, or both, are labeled. Examples of labels include, but are not limited to, a detectable label such as, for example, a radioactive label (e.g., a radioisotope), a fluorescent label (e.g., free fluorophores can be coupled via free COOH-groups), succinimidyl (NHS-) esters, amines from lysine residues, or thiols from cysteine residues, maleimides and cyanine dyes suitable for coupling to thiol containing groups such as those contained in cysteine residues), an enzyme (e.g., an enzyme the activity of which results in a change in a detectable signal, e.g., a change in color or emission, e.g., fluorescence), a proximity-based signal generating label (e.g., a FRET component), a homogeneous time resolved fluorescence (HTRF) component, a luminescent oxygen channeling assay (LOCI) component, biotin, avidin, or another functionally similar substance, an antibody (e.g., a primary or a secondary antibody), or a portion thereof (e.g., an antigen binding portion of an antibody).

Suitable energy absorbing FRET components include fluorophores (e.g., NDB, dansyl, pyrene, anthracene, rhodamine, fluorescein and indocarbocyanine, and their derivatives). Dyes useful as energy absorbing FRET donor/acceptor pairs include indocarbocyanine/indocarbocyanine, (e.g., fluoresceino/rhodamine, NBD N-(7-nitrobenz-2-oxa-1,3-diazol-3-yl)/rhodamine, fluorescein/eosin, fluorescein/erythrosin, dansyl/rhodamine, acridine orange/rhodamine, pyrene/fluorescein, 7-amino-actinomycin-D/fluorescein, 7-aminoactinomycin-D/R-phycoerythrin, fluorescein/R-phycoerythrin, ethidium monoazide/fluorescein, and ethidium monoazide/R-phycoerythrin. In some exemplary embodiments, the dye is selected from the group consisting of the Cy family of dyes (Amersham BioSciences), such as Cy 3.5 and Cy 5.5, and the Alexa family of dyes (Molecular Probes), such as Alexa 647 and Alexa 568. Many of these dyes are commercially available or can be synthesized using methods known to those of ordinary skill in the art.

The sensors can be used to detect a wide range of physiological analyte concentrations (e.g., concentrations ranging from 0.5 to 18 mg/ml in the case of glucose).

In another aspect, an embodiment of the invention features a method for evaluating a carbohydrate in a sample that is carried out by first contacting the sample with a specific binding pair that includes a first binding member and a second binding member. The first binding member includes a mutant ConA as taught in the present application coupled to a first energy absorbing FRET component, and the second binding member including a glycoconjugate that further includes a carbohydrate and a second energy absorbing FRET component. The excited state energy level of the first energy absorbing FRET component overlaps with the excited state energy level of the second energy absorbing FRET component, and the mutant ConA and the glycoconjugate can reversibly bind to each other such that carbohydrate present in the sample can displace the glycoconjugate and reversibly bind to the mutant ConA. The extent to which non-radiative fluorescence resonance energy transfer occurs between the first energy absorbing FRET component and the second energy absorbing FRET component is then evaluated. This evaluation reflects the presence of carbohydrate in the sample and correlates with its amount. The evaluation can be made in the presence of the glycoconjugate displaced by the carbohydrate and the mutant ConA reversibly bound to the carbohydrate.

Energy transfer can be evaluated in numerous ways. For example, it can be evaluated by measuring one or more of: donor quenching, donor lifetime (e.g., a decrease in donor excited lifetime), sensitized acceptor emission, or fluorescence depolarization. It can also be measured by determining the ratio of two parameters, such as the ratio of: a donor parameter to an acceptor parameter (e.g., the ratio of donor to acceptor fluorescence, or depolarization of fluorescence relative to excitation); a donor parameter to a donor parameter (e.g., the ratio of donor to donor fluorescence, or depolarization of fluorescence relative to excitation); an acceptor parameter to an acceptor parameter (e.g., the ratio of acceptor fluorescence or depolarization of fluorescence relative to excitation). For example, (and regardless of whether the method is carried out ex vivo, or in vivo) the evaluation can include measuring energy transfer as a function of fluorescence intensities of the first energy absorbing FRET component and the second energy absorbing FRET component. The evaluation can also include a comparison between the extent to which non-radiative fluorescence resonance energy transfer occurs between the first and second energy absorbing FRET components and a FRET value obtained from a calibration step.

In the event the detectable label is a homogeneous time resolved fluorescence (HTRF) component, the evaluation will include measuring energy transfer as a function of fluorescence intensities of a first and second energy absorbing HTRF component. Similarly, in the event the detectable label is a luminescent oxygen channeling assay (LOCI) component, the evaluation will include measuring energy transfer as a function of the photochemical reaction of a first energy absorbing LOCI component and a second chemiluminescence-producing LOCI component.

In exemplary embodiments, either the first or second energy absorbing FRET component is a fluorophore (e.g., fluorescein, rhodamine, BODIPY, a cyanine dyes, or a phycobiliprotein). For example, a mutant ConA as described herein can be labeled with a fluorophore, and the glycoconjugate can be labeled with a fluorophore in the non-radiative fluorescence resonance energy transfer process. A mutant ConA can also be labeled with a fluorophore that is the acceptor and the glycoconjugate can be labeled with a fluorophore that is the donor in the non-radiative fluorescence resonance energy transfer process. For example, the first member of a specific binding pair can be fluorophore-labeled mutant ConA, and the second member of the specific binding pair can be fluorophore-labeled glycosylated serum albumin that binds to mutant ConA. Here, the non-radiative fluorescence resonance energy transfer can be determined by measuring the ratio of the light emissions attributable to the two fluorophores.

Another aspect of the invention includes an in vivo method for evaluating a carbohydrate (e.g., glucose) in a subject. The method can be carried out by placing a first binding member and a second binding member (i.e., a sensor) in contact with the carbohydrate in the body fluids of the subject (e.g., the sensor can be introduced into an organ or vessel where it would be exposed to glucose). Once in place, the presence and/or amount of the carbohydrate can be monitored without further invasive procedures. For example, a sensor can be placed in, on, or under the subject's skin and glucose can be evaluated by illuminating the sensor at the excitation wavelength of, e.g., an energy absorbing FRET donor. Energy transfer between two energy absorbing FRET components can be detected by a fluorimeter (e.g., a filter based or a monochromater based fluorimeter) that measures, for example, the ratio of fluorescence intensities at the two emission maxima wavelengths of the energy absorbing FRET components, or the quenching of the energy absorbing donor fluorescence at its emission maximum as a function of glucose concentration.

The first binding member can include a mutant ConA as described herein coupled to a first energy absorbing FRET component, and the second binding member can include a glycoconjugate that includes a carbohydrate and a second energy absorbing FRET component. The excited state energy levels of the first and second energy absorbing FRET components can overlap, and the mutant ConA and the glycoconjugate can reversibly bind one another (in which case, carbohydrate present in the sample would displace the glycoconjugate and reversibly bind to the mutant ConA). The extent or degree to which non-radiative fluorescence energy is transferred between the first and second energy absorbing FRET components can then be measured or monitored non-invasively.

As with methods carried out ex vivo, energy transfer can, for example, be evaluated by measuring one or more of: donor quenching, donor lifetime (e.g., a decrease in donor excited lifetime), sensitized acceptor emission, or fluorescence depolarization. It can also be measured by determining the ratio of two parameters, such as the ratio of: a donor parameter to an acceptor parameter (e.g., the ratio of donor to acceptor fluorescence, or depolarization of fluorescence relative to excitation); a donor parameter to a donor parameter (e.g., the ratio of donor to donor fluorescence, or depolarization of fluorescence relative to excitation); an acceptor parameter to an acceptor parameter (e.g., the ratio of acceptor to acceptor fluorescence, or depolarization of fluorescence relative to excitation).

Preferably, the sensor is positioned to evaluate a carbohydrate analyte (such as monosaccharides, disaccharides, a polysaccharide, glucose, a carbohydrate that is a component of another molecule or a supramolecular structure (e.g., a macromolecule), or combination thereof) in the subject's subcutaneous body fluid, intracutaneous body fluid, or blood.

In another aspect, the invention features a sensor for non-invasively monitoring a carbohydrate (e.g., glucose) in a subject (i.e., the subject's skin does not have to be punctured each time a glucose level is obtained). The sensor can also be used to evaluate carbohydrates ex vivo (e.g., in a blood sample obtained from a subject). The sensor includes a specific binding pair that includes a first binding member and a second binding member, the first binding member including a mutant ConA of the present invention coupled to a first energy absorbing FRET component, and the second binding member including a glycoconjugate that includes a carbohydrate and a second energy absorbing FRET component. The excited state energy levels of the first and second energy absorbing FRET components overlap and the mutant ConA of the present invention and the glycoconjugate reversibly bind one another. Thus, carbohydrate present in the sample can displace the glycoconjugate and reversibly bind to the mutant ConA of the present invention. Energy transfer can be evaluated as described above.

In vivo methods can be modified to provide positive feedback. For example, when glucose is monitored and found to be above an acceptable range, insulin can be administered (e.g., by an implanted pump) to lower the high level. In contrast, when glucose is below an acceptable range, a signal or alarm can be triggered to alert the subject (who can then ingest food or drink to raise the low level).

An energy absorbing FRET component, as used herein, is a substance that can either be a donor or an acceptor in the process of non-radiative energy transfer. Both the donor and the acceptor absorb energy. The function of the donor is to absorb energy at a first wavelength and transmit the absorbed energy via non-radiative energy transfer to the acceptor molecule. The function of the acceptor is to absorb the transmitted energy from the donor. The absorbed energy can be dissipated in a number of ways, for example, by emission of the energy at a second wavelength, dissipation as heat energy, or transfer of energy to the surroundings. Absorption by the acceptor can be measured by an acceptor parameter, e.g., sensitized acceptor emission or a donor parameter, e.g. donor fluorescence quenching. Requirements of the energy absorbing FRET components are that there is sufficient energy state overlap between the two in order for non-radiative energy transfer to occur. Furthermore, non-radiative energy transfer occurs only if the two are in close proximity (half energy transfer between a single donor and acceptor molecule occurs when the intermolecular distance is R₀).

An “energy absorbing FRET donor” is a substance that absorbs energy at a first wavelength. The absorbed energy creates an excited state in the donor. The donor can leave the excited state by emitting energy at an emission wavelength, by dissipating the energy in the form of heat, or by transmitting the absorbed energy via non-radiative energy transfer to an energy absorbing FRET acceptor. Accordingly, an “energy absorbing FRET acceptor” is a substance that absorbs the non-radiative energy transferred from the energy absorbing FRET donor. The absorbed energy creates an excited state in the acceptor, which the acceptor can leave by emitting the absorbed energy at a second wavelength, dissipating energy as heat, or transferring energy to its surroundings.

A first component “specifically binds” a second when the first component binds the second with a substantially higher affinity (e.g., with 50% greater affinity) than it binds a related component or moiety.

An interaction is “reversible” if it can proceed in either direction. A reversible reaction can consist, for example, of a forward reaction in which a glycoconjugate binds to a mutant ConA as taught herein and a reverse reaction in which the glycoconjugate is released from the mutant ConA. Reversible reactions should occur under the conditions (e.g., physiological conditions) in which a carbohydrate is evaluated.

One skilled in the art will appreciate further features and advantages of the invention based on the above-described embodiments. Accordingly, the invention is not to be limited by what has been particularly shown and described, nor by the examples set forth below, except as indicated by the appended claims. All publications and references cited herein are expressly incorporated herein by reference in their entirety.

### EXAMPLES

I. Expression and Purification of Recombinant Mutant ConA

A. Cloning Mature Mutant ConA Coding Region

Due to the post-translational modifications necessary for producing “mature” ConA, cloning the DNA coding region for “mature” ConA is challenging. ConA maturation requires a series of proteolytic digestions followed by transpeptidation of the N-terminal and C-terminal halves of a non-functional precursor (pre-pro-ConA) (Carrington, D. M., et al. Polypeptide ligation occurs during post-translational modification of concanavalin A. Nature, 1985. 313(5997): p. 64-7). From a cloning perspective, the result is a primary amino acid sequence that does not correspond to the predicted amino acid sequence derived from the genomic ConA coding region. This prevents direct cloning of the “mature” ConA coding region from natural DNA sources.

In lieu of directly cloning the “mature” ConA coding region suitable for expression, the “mature” ConA DNA sequence was assembled based on genomic, precursor DNA sequences. Construction of the mature ConA coding region required isolating and rearranging those sections of preConA DNA which code for the mature ConA primary amino acid sequence. SEQ ID NOS: 3 and 4, respectively show the corresponding amino acid sequence of mature C. ensiformis (AA seq from Carrington, et al.) and amino acid sequence of mature C. gladiata (AA seq from Yamauchi, et al. FEBS Lett. 260:127 1990). The two deduced “mature” ConA DNA sequences were used to design and construct recombinant ConA expression systems.

B. Construction of ConA cDNA

i. Gene Synthesis Technology

Due to the extensive DNA rearrangement required to design a mature ConA coding region, there were a limited number of methods to construct a “mature” ConA cDNA for bacterial expression. One method utilized gene synthesis technology in which single nucleotides were ligated chemically according to a predesigned DNA sequence. This procedure was analogous to methods used in the synthesis of oligonucleotides. There were several benefits in using gene synthesis as a means for cDNA construction. First, the ease in which coding regions for chimeric proteins (e.g. mature ConA) could be synthesized. Second, coding regions could be optimized for codon usage in any host expression system to maximize recombinant protein expression. Finally, restriction sites could be engineered anywhere for future cloning purposes.

Gene synthesis of mature ConA was performed by the company GeneArt (Germany). Several modifications were made to the ConA coding region. First, the synthesized ConA gene was optimized for codon usage in E. coli. Second, NcoI and BamHI restriction sites were engineered at 5′ and 3′ ends, respectively, for cloning into the bacterial expression vector pET1 Sb. Finally, the secretion signal sequence from the E. coli outer membrane protein (ompA) was engineered at the 5′ end of the ConA coding sequence.

ii. cDNA Cloning from Jack Bean

Another method for constructing a mature ConA cDNA, was through direct cloning of the precursor ConA coding region from Canavalia ensiformis beans (jack beans). This was a multistep cloning process requiring the synthesis of pre-ConA cDNA from isolated jack bean RNA, cloning of preConA cDNA, and genetic rearrangement of the pre-ConA coding region by PCR to generate the mature ConA coding region. The result would be a single cDNA clone that codes for mature ConA identical to that obtained with gene synthesis.

(a) Synthesis and Purification of Jack Bean Total RNA and cDNA

Mature ConA cDNA was synthesized from total jack bean cDNA derived from purified RNA. Immature jack beans (0.5 kg) were harvested from ˜6 week old Canavalia ensiformis plants (Plantwise Enterprises). The beans were rapidly frozen in liquid nitrogen, to preserve the beans and eliminate all RNase activity, and stored at −80° C. A single jack bean (˜1.8g) was crushed using a pre-chilled mortar and pestle pre-treated with RNAZap from Ambion. A 1 ml pipette tip was used to scoop ˜⅓ of the crushed meal and transferred to a sterile microfuge tube (˜250 ul in volume). Total RNA was isolated from the meal using RNAqueous total RNA isolation kit and Plant Isolation Aid (Ambion) according to manufacturers conditions. Approximately 50 ug of total jack bean RNA was isolated by this method.

(b) Isolation and Cloning of Precursor ConA cDNA (Pre-Pro ConA)

Next, pre-ConA cDNA was purified and cloned into a conventional sequencing vector. An aliquot of purified total RNA (4 ug) was first reverse transcribed with MMLV reverse transcriptase using Retroscript (Ambion) to generate an aliquot of total jack bean cDNA. Gene specific isolation of the pre-pro ConA cDNA was then achieved by polymerase chain reaction (PCR). Three gene specific forward primers and one reverse primer, based on the pre-pro ConA sequence (Genbank), were designed using the Primer Premier software suite (Biosoft International) (Table. 1). Three PCR reactions using each primer pair and 5ul of total cDNA were set up using an Eppendorf Mastercycler and employing the Touchdown PCR conditions outlined in Table. 2. PCR reaction efficiencies were assessed by agarose gel electrophoresis. PCR products corresponding to the correct approximate molecular weight of pre-pro ConA cDNA (˜950 bp) were band purified by preparative gel electrophoresis and isolated using Zymoclean (Zymo Research). Gel purified PCR products were cloned into the sequencing vector pCR2.1 using TOPO TA cloning kit for sequencing (Invitrogen).

TABLE 1
Pre-pro ConA PCR primers

| | | |
|---|---|---|
| Direction | Name | Sequence |

| | | |
|---|---|---|
| Sense | 5′preConA1 | 5′ATTGTAGCAAGCAGCACTAC3′ |
| | | SEQ ID NO:29 |
| Sense | 5′preConA2 | 5′TAGCAAGCAGCACTACTAGTG3′ |
| | | SEQ ID NO:30 |
| Sense | 5′preConA3 | 5′GCAAGCAGCACTACTAGTGA3′ |
| | | SEQ ID NO:31 |
| Anti- | 3′preConA | 5′GAGATTATTATGGTACATGGATGA3′ |
| sense | | SEQ ID NO:32 |

TABLE 2
Pre-pro ConA PCR conditions

| Stage | Temperature | Time (minutes) | # of cycles |
|---|---|---|---|
| Initial denature | 94° C. | 2.0 | 1 |
| Denature | 94° C. | 0.5 | 5 |
| Annealing | 51° C. | 0.5 | 5 |
| Extension | 72° C. | 1.0 | 5 |
| Denature | 94° C. | 0.5 | 5 |
| Annealing | 48° C. | 0.5 | 5 |
| Extension | 72° C. | 1.0 | 5 |
| Denature | 94° C. | 0.5 | 25 |
| Annealing | 45° C. | 0.5 | 25 |
| Extension | 72° C. | 1.0 | 25 |
| Extension | 72° C. | 5.0 | 1 |
| Hold | 4° C. | overnight | 1 |

(c) Verification of Pre-Pro ConA cDNA Sequence

Next, DNA sequencing analysis was employed to ensure the cloned PCR products corresponded to the published pre-pro ConA sequence. Two independent clones containing the PCR product were isolated and analyzed by DNA PCR cycle sequencing (University of Massachusetts Medical School Nucleic Acid Facility). Two sequencing primers (M13 universal and M13 reverse) were used in separate sequencing reactions to sequence the entire cloned DNA. DNA sequence data from the reactions were received as ABI (Applied Biosystems) chromatograms and analyzed using the Sequencher software suite (Gene Codes Corporation). The DNA sequence of the cloned PCR products completely matched the Genbank published sequence using the BLAST DNA alignment algorithm (NCBI).

iii. “Mature ” ConA cDNA Synthesis by SOE PCR

With the pre-pro ConA cDNA cloned, the mature ConA coding region was generated using a specific PCR method known as gene splicing overlap extension (SOE PCR). SOE PCR is a PCR procedure used for the creation of novel genes including chimeric proteins (Warrens, A. N., et al. Gene, 1997. 186(1): p. 29-35). With SOE PCR, PCR primers were specifically designed to unique regions of a target sequence to add, delete, or rearrange any portion of the DNA. This type of genetic rearrangement required two sequential PCR reactions and four PCR primers, two of which were almost completely complementary. The first series of PCR reactions produced DNA products that were complementary within a specific region that creates the chimera. To endfill the uncomplemented regions, the annealed PCR products were used as the template for the second PCR reaction to complete the final chimeric product.

To use SOE PCR for synthesizing mature ConA cDNA, the mature DNA sequence was compared to the pre-pro ConA sequence to devise a PCR primer strategy. As stated above, one of the primary modifications of preConA maturation is a transpeptidation reaction which entails the switching and re-ligation of the C-terminal and N-terminal halves of the protein. The initial strategy was to deduce those regions of pre-pro ConA involved in the transpeptidation reaction at the DNA level. The coding region from B1 to B2 is the N-terminal half while the sequence from A1 to A2 represents the C-terminal half. Four PCR primers were design using Primer Premier that are complementary to those regions involved in the transpeptidation reaction (Table. 3). One primer pair was directed towards the N-terminal half of mature ConA (ConApt1 (C and D)) while the second primer pair generated the C-terminal half (ConApt2 (A and B)). The overlapping primers (ConApt1(D) and ConApt2(A), Table 3) facilitated the synthesis of the final mature ConA product by mimicking the transpeptidation reaction at the DNA level.

TABLE 3

Primer pair sequences for ConA SOE
PCR (1^{st} round)

| | | |
|---|---|---|
| Direction | Name | Sequence |

| | | |
|---|---|---|
| Sense | ConApt1(C) | 5′GCCGATACTATTGTTGCTGTTGAATTG |
| | | GAT3′ |
| | | SEQ ID NO:33 |
| Anti- | ConApt1(D) | 5′GAAATGGAGTGCATTTGTCTCATGTGT |
| Sense | | TGAATTGCTCTTCAACTTAGAAGTAAAAG |
| | | ACCA3′ |
| | | SEQ ID NO:34 |
| Sense | ConApt2(A) | 5′TGGTCTTTACTTCTAAGTTGAAGAGCA |
| | | ATTCAACACATGAGACAAATGCACTCCAT |
| | | TTC3′ |
| | | SEQ ID NO:35 |
| Anti- | ConApt2(B) | 5′TCAATTTGCATCAGGGAAGAGTCCAAG |
| Sense | | GAGCCT3′ |
| | | SEQ ID NO:36 |

The conditions used for SOE PCR of the mature ConA cDNA are outlined above. Purified pCR2.1-preConA was used as the template in the first series of PCR reactions. The PCR products (˜350 bp each) from each reaction were purified by agarose gel electrophoresis and extracted using Zymoclean (Zymo Research). The second PCR reaction (primers ConApt1(C) and ConApt2(B) plus the annealed PCR product as template) resulted in a ˜700 base pair product, approximately the predicted size for mature ConA cDNA. The PCR product was purified by agarose gel electrophoresis, extracted using Zymoclean (Zymo Research) and cloned into the pCR2.1 sequencing vector (TOPO TA cloning kit for sequencing, Invitrogen). Confirmation of successful mature ConA cDNA synthesis was determined by DNA PCR cycle sequencing (University of Massachusetts Medical School Nucleic Acid Facility). Two sequencing primers (M13 universal and M13 reverse) were used in separate sequencing reactions to sequence the entire cloned DNA. DNA sequence data from the reactions were received as ABI (Applied Biosystems) chromatograms and analyzed using Sequencher software suite (Gene Codes Corporation). The DNA sequence of the cloned PCR products completely match the mature ConA sequence defined in herein using the BLAST2 DNA alignment algorithm.

TABLE 4
Primer pair sequences for mutant ConA SOE

| Direc- | | |
|---|---|---|
| tion | Mutation | Sequence |
| Sense | Asp/ | 5′cacatcatctataactctgttTGTaagaga |
| | Gly58→*Cys | ctaagtgctgttgtttcttatcctaacgct3′ |
| | | SEQ ID NO:37 |
| Anti- | Asp/ | 5′agcgttaggataagaaacaacagcacttag |
| Sense | Gly58→*Cys | tctcttACAaacagagttatagatgatgtg3′ |
| | | SEQ ID NO:38 |
| Sense | Asp/ | 5′cacatcatctataactctgttCCTaagaga |
| | Gly58→*Pro | ctaagtgctgttgtttcttatcctaacgct3′ |
| | | SEQ ID NO:39 |
| Anti- | Asp/ | 5′agcgttaggataagaaacaacagcacttag |
| Sense | Gly58→*Pro | tctcttAGGaacagagttatagatgatgtg3′ |
| | | SEQ ID NO:40 |
| Sense | Asp/ | 5′cacatcatctataactctgttAATaagaga |
| | Gly58→*Asn | ctaagtgctgttgtttcttatcctaacgct3′ |
| | | SEQ ID NO:41 |
| Anti- | Asp/ | 5′agcgttaggataagaaacaacagcacttag |
| Sense | Gly58→*Asn | tctcttATTaacagagttatagatgatgtg3′ |
| | | SEQ ID NO:42 |
| Sense | Glu192→*Gln | 5′tctgctgtggtggccagctttCAAgctacc |
| | | tttacatttctcataaaatcacccgactct3′ |
| | | SEQ ID NO:43 |
| Anti- | Glu192→*Gln | 5′agagtcgggtgattttatgagaaatgtaaa |
| Sense | | ggtagcTTGaaagctggccaccacagcaga3′ |
| | | SEQ ID NO:44 |
| Sense | Glu192→*Pro | 5′tctgctgtggtggccagctttCCAgctacc |
| | | tttacatttctcataaaatcacccgactct3′ |
| | | SEQ ID NO:45 |
| Anti- | Glu192→*Pro | 5′agagtcgggtgattttatgagaaatgtaaa |
| Sense | | ggtagcTGGaaagctggccaccacagcaga3′ |
| | | SEQ ID NO:46 |
| Sense | Glu192→*Cys | 5′tctgctgtggtggccagctttTGTgctacc |
| | | tttacatttctcataaaatcacccgactct3′ |
| | | SEQ ID NO:47 |
| Anti- | Glu192→*Cys | 5′agagtcgggtgattttatgagaaatgtaaa |
| Sense | | ggtagcACAaaagctggccaccacagcaga3′ |
| | | SEQ ID NO:48 |
| Sense | Asn118→*Cys | 5′TCATGGTCTTTTACTTCTAGTTGAAGAGC |
| | | TGTTCAACACATGAGACAAATGCACTCCAT3′ |
| | | SEQ ID NO:49 |
| Anti- | Asn118→*Cys | 5′ATGGAGTGCATTTGTCTCATGTGTTGAACA |
| Sense | | GCTCTTCAACTTAGAAGTAAAAGACCATGA3′ |
| | | SEQ ID NO:50 |
| Sense | His121→*Tyr | 5′acttctaagttgaagagctgttcaacaTAT |
| | | gagacaaatgcactccatttcatgttcaac3′ |
| | | SEQ ID NO:51 |
| Anti- | His121→*Tyr | 5′gttgaacatgaaatggagtgcatttgtctc |
| Sense | | ATAtgttgaacagctcttcaacttagaagt3′ |
| | | SEQ ID NO:52 |
| Sense | His121→*Cys | 5′acttctaagttgaagagctgttcaacaTGT |
| | | gagacaaatgcactccatttcatgttcaac3′ |
| | | SEQ ID NO:53 |
| Anti- | His121→*Cys | 5′gttgaacatgaaatggagtgcatttgtctc |
| Sense | | ACAtgttgaacagctcttcaacttagaagt3′ |
| | | SEQ ID NO:54 |
| Sense | His121→*Pro | 5′acttctaagttgaagagctgttcaacaCCT |
| | | gagacaaatgcactccatttcatgttcaac3′ |
| | | SEQ ID NO:55 |
| Anti- | His121→*Pro | 5′gttgaacatgaaatggagtgcatttgtctc |
| Sense | | AGGtgttgaacagctcttcaacttagaagt3′ |
| | | SEQ ID NO:56 |

iv. Mutant ConA cDNA Synthesis by SOE PCR

Similar conditions were used for SOE PCR of the mutant ConA cDNA. Table 4 contains a list of primer pairs used to make the mutant ConA's. Additional mutations were added to previously constructed plasmids. For example, the double mutants were built off single mutants, the triple mutants were built off of double mutants, and the quad mutants were built off of the triple mutants.

C. Expression of Mutant ConA Proteins

i. Selection of Bacterial Expression System

Any suitable expression system can be used. Useful expression systems include e.g., cell free translation systems, as well as, cell based translation systems (e.g., mammalian, yeast, insect, bacterial). Bacterial expression systems provide for both soluble and insoluble expression. A specific example of a suitable expression system includes an E. coli based system, which directs the expressed proteins into inclusion bodies. Inclusion bodies can be utilized for the enrichment of expressed recombinant protein. By using specific growth conditions and expression system components that force synthesized recombinant proteins into inclusion bodies, the recombinant protein of interest was easily harvested by simple, centrifugal fractionation procedures.

To ensure the production of inclusion bodies composed solely of insoluble mutant ConA, the secretion signal sequence of the E. coli outer membrane protein (ompA) was used to facilitate mutant ConA enrichment. The ompA DNA signal sequence was ligated to the 5′ end of the mature mutant ConA sequence by both gene synthesis and DNA recombinant technology to facilitate mutant ConA purification.

The pET15b vector, which contains an ampicillin resistance gene was predominantly used for the cloning and expression of wild-type ConA (gConA). Mutant ConA proteins, more fully described below, were cloned and expressed using the pET24b plasmid, which carries a kanamycin resistance gene.

ii. Bacterial Expression Conditions

(a) Selection of E. coli Strain

Two common E. coli strains for T7 RNA polymerase-based expression systems, BL21(DE3) and BL21(DE3)pLys were used. Expression of ConA using BL21(DE3) and BL21(DE3)pLys strains of E. coli were compared to optimize for levels of expression. Small-scale bacterial expression (<50 ml) was used to express ConA in BL-21 (negative control), BL21(DE3) and BL21(DE3)pLys. Isolated inclusion bodies were resuspended in SDS sample buffer and boiled at 95° C. for 5 minutes and analyzed on SDS-PAGE. Ten μl of sample extract was loaded in each gel well. Since expression levels of ConA were highest in BL21(DE3), this bacterial strain was selected for subsequent expression of mutant ConAs.

(b) Specific Induction of ConA Expression in DE3

Two BL21(DE3) clones expressing rConA were selected to characterize specific induction by isopropyl β-D-thiogalactopyranoside (IPTG). Small-scale bacterial expression (<50 ml) was used to express mutant ConA in two BL21(DE3) clones, DE3-1 and DE3-2. Isolated inclusion bodies were resuspended in SDS sample buffer and boiled at 95° C. for 5 minutes and analyzed on SDS-PAGE. Since both DE3-1 and DE3-2 exhibited IPTG dependent induction of mutant ConA expression, both clones were used for subsequent expression of wild-type recombinant ConA from C. ensiformis.

(c) Effect of Temperature on Mutant ConA Expression

Localization of recombinant ConA in soluble and insoluble (inclusion bodies) fractions during expression in E. coli is dependent on temperature (Min, W., Emulation of the Post-translational Processing of Concanavalin A by Recombinant DNA Manipulations, in School of Biological Sciences. 1992, University College of Swansea: Swansea. p. 255). To select the optimal temperature for mutant ConA expression, small-scale bacterial cultures (<50 ml) were induced at two temperatures, 30° C. and 37° C. Subsequent purification efforts utilized 37° C. for bacterial growth and induction, and focused on proper refolding and affinity purification of expressed mutant ConA.

D. Production and Purification of Recombinant Mutant ConA

i. Preparation of Induction Cultures

Two induction cultures were grown over a 48-hour period. The first culture consisted of the inoculation of single 25 ml 2XYT/Kanamycin culture with either a single bacterial colony (BL21(DE3)) containing a plasmid containing, for example, SEQ ID NOS: 5, 7, 9, 11, 13, 15, 17, 19, 21, 23, or 25 or directly from frozen bacterial glycerol stock containing the plasmid was shaken overnight at 37° C. in an incubator.

ii. Induction

To induce expression of mutant ConA, 6 ml of overnight culture was used to inoculate 1 L of 2xYT/Kanamycin culture (IL per 2 L flask-4 L total) pre-warmed 37° C. The culture then grows for 1.75 hours at 37° C. in a shaking incubator (300 rpm). For maximal protein expression, bacterial cultures were induced during the logarithmic phase of the growth cycle. The optical density of the culture at 600 nm was determined with a spectrophotometer. Typically, optical density of a logarithmically growing culture is between 0.6 and 0.8. Once the culture has reached the appropriate optical density, 119 mg of isopropyl β-D-thiogalactopyranoside per liter of log phase culture was added to a final concentration of 0.5 mM. The induced culture incubates at 37° C. in shaking incubator for additional 3 hours. At the end of the induction period, the culture was centrifuged and the bacterial pellets stored overnight at −80° C.

iii. Inclusion Body Purification

The frozen bacterial pellets were resuspended in 400 ml of ConA lysis buffer (20 mM MOPS, 1M NaCl, 5 mM EDTA, 0.5% Triton X-100, 0.01% sodium azide, 1 mg/ml lysozyme) to release the inclusion bodies. 25 ml of the resuspended pellet was aliquoted into eight 35 ml Oak Ridge tubes. To shear residual chromosomal DNA and lyse any remaining intact cells, lysates were sonicated for 1 min. The insoluble protein fraction was subsequently isolated by centrifuging the lysates at high speeds (17,500 rpm) at 4° C. for 20 minutes.

To further purify the inclusion body fraction, the insoluble pellet underwent several washing steps to remove any contaminating soluble proteins and other cellular debris. Inclusion body pellets were resuspended in 100 ml of ConA lysis buffer (without lysozyme/DNasel) via brief sonication (30 sec). The resuspended pellets were centrifuged at 17,500 rpm at 4° C. This process was repeated 2× more with ConA lysis buffer (3× total). To remove detergent from the inclusion body pellet, the pellet was washed with 100 ml of Con A wash buffer (ConA lysis buffer without Triton X-100). Finally, to prepare for the denaturation/renaturation step of the purification procedure, EDTA was removed to allow the refolded rConA to coordinate Mn²⁺ and Ca²⁺ for proper function. To achieve this, the inclusion body pellet was washed a final time in ConA storage buffer (20 mM MOPS, 1M NaCl, 1 mM manganese chloride, 1 mM Calcium chloride, pH 7.0). The purified inclusion body pellets were frozen in liquid nitrogen and stored at −80° C.

iv. Denaturation/Renaturation Recombinant Mutant ConA

Purified inclusion bodies were thoroughly solubilized and mutant ConA was allowed to refold slowly. Inclusion body pellets were solubilized and mutant ConA denatured by adding 20 ml ConA denaturing buffer (containing 6M guanidine hydrochloride) per liter of culture followed by brief (10-20 sec.) sonication. The partially solubilized pellets were incubated overnight at 4° C. with slow rotation. At this point, the suspension was centrifuged at 17,500 rpm for 20 minutes at 4° C. to remove any insoluble material.

To initiate refolding of mutant ConA, the supernatant was slowly diluted 30-fold at 4° C. overnight using a syringe pump. The flow rate from the syringe pump was about 100 μl per minute with gentle stirring to allow thorough mixing of denatured mutant ConA in the dilution buffer to ensure proper refolding and the formation of intact tetramers.

v. Affinity Purification

The clarified protein solution was loaded onto a 40 ml Sephadex G75 column pre-equilibrated with ConA metals buffer at a flow rate of approximately 2.25 ml/min. The column was immediately washed 2× with 400 ml ConA metals buffer. Bound protein was eluted three times by resuspending the matrix in 100 ml ConA elution buffer (total volume—40 ml, 30 ml, 30 ml) containing 20 mM methyl α-D-mannopyranoside. The protein concentration of the pooled eluate was calculated (see below) and stored at 4° C.

II. Purification of Natural ConA

Wild-type ConA was purified from natural sources using a modification of the method of Cunningham, et. al. A 10 mg/ml solution of natural ConA was re-suspended in 1% ammonium bicarbonate, pH 8.0 at 37° C. for 18 hours. The suspension was centrifuged at 12 k rpm and supernatant loaded on 1 ml Sephadex G-75 column. Twenty (20)μl from each stage was run on 10% Bis-Tris acrylamide gel and stained with colloidal blue (Simply Blue, Invitrogen). This method resulted in enrichment for homotetrameric ConA in the purified supernatant. This differential precipitation technique resulted in ˜93.5% pure homotetrameric ConA when combined with a Sephadex G-75 affinity chromatography step to ensure purification of active ConA tetramer.

Purification of full-length, wild-type natural ConA monomers was also accomplished through the complete denaturation and reassembly of wild-type natural ConA homotetramers using size exclusion chromatography. Extremely harsh biochemical conditions are necessary for the disassembly and denaturation of ConA tetramers (Auer, H. E. and T. Schilz, Int J Pept Protein Res, 1984.24(6): p. 569-79; Auer, H. E. and T. Schilz,. Int J Pept Protein Res, 1984. 24(5): p. 462-71; Huet, M., Eur J Biochem, 1975. 59(2): p. 627-32). ConA tetramers assemble in a pH dependent manner, forming stable tetramers between pH 7.0-7.5. Multimeric complexes consisting of high molecular weight aggregates occur at pH's greater than 7.5. Supernatant from NH₄HCO₃ precipitation was dialyzed against 8M Urea denaturing buffer, and the eluent concentrated to a final volume of 5 ml. The linear ConA polypeptide chains were purified to near homogeneity by size exclusion chromatography (Abe, Y., M. Iwabuchi, and S. I. Ishii, Biochem Biophys Res Commun, 1971. 45(5): p. 1271-8.).

Two (2) ml of concentrate was loaded on a Sephacryl S-100 column pre-equilibrated with 8M Urea denaturing buffer. Fractions corresponding to ConA 26 kDa polypeptide were collected and pooled (50-fractions, 1 ml/each, flow rate of 0.5 ml/mi n). The pooled fractions revealed a 1.7 fold enrichment representing 90% of the total protein as shown by SDS-PAGE analysis. The remaining protein represents the 12 kDa fragment.

To reassemble ConA tetramers, denatured samples were diluted 30 fold in renaturation buffer (pH7.0 with Mn²⁺ and Ca²⁺) and purified by affinity chromatography (Sephadex G-75). Tetramers purified by this protocol were composed solely of 26 kDa monomer with no detectable levels of contaminating protein bands as demonstrated by gel electrophoresis.

This method was not only applicable to the purification of wild-type ConA but may be used, generally, to purify lectins from various sources, including Concanavalin A from recombinant sources.

III. Protein Characterization

A. Concentration Determination and Purity Analysis

i. UV Analysis

Two analytical assays were conducted to determine the protein concentration, percentage yield, and purity of the purified material, for the mutant ConA dimers. To monitor the purification process, aliquots were removed at all stages of purification starting at the inclusion body purification steps. To determine the protein concentration of the mutant ConA eluates, the absorbance of undiluted eluate at wavelength 280 nm was determined using a spectrophotometer. The values generated were used to calculate the concentration using the extinction coefficient for ConA. (OD₂₈₀ ˜1.14=1 mg/ml ConA).

Percentage yield was calculated to determine amount of recoverable mutant ConA during the purification procedure. To calculate this value, the concentration of the eluate was divided by the concentration of the starting material. After the refolding and clarification steps, the absorbance of undiluted, refolded mutant ConA at 280 nm was determined and the concentration of the starting material calculated as described above. The percentage yield was computed by calculating the ratio of the eluate and total mutant ConA concentrations

ii. SDS PAGE

As a final analytical step, the purity of mutant ConA was determined both qualitatively and quantitatively. Qualitative analysis entailed visualizing the amount of 26 kD mutant ConA monomer present by SDS-PAGE.

All mutant ConA solutions were diluted in equal volumes of Sample Buffer (2X). The sample solutions were then heated for 10±1 minutes at 95±5° C. After cooling to room temperature, the samples were loaded on the gel. Analytical tests were conducted using NuPAGE® 10% Bis-Tris gels in the Xcell SureLock® Mini-Cell. The gels were loaded with 20 μL of the samples (5 μL of the marker). The gel rinsing, staining, and destaining steps all required 100 mL of the respective solutions. The gels were scanned and quantitated using the Bio-Rad Model Gel Doc® EQ Imaging System.

The final concentration of the reducing agent in the sample solution was 1×. The marker used was Invitrogen Multimark molecular weight markers, that consists of thirteen protein bands. The NuPAGE® running buffer with 2-Morpholinoethanesulfonic acid (MES) was used. The gel was run at a voltage of 200V. The run time was 35 minutes. In the SDS removal step, 50 of dH₂O was added to the gel and microwaved for 2.5 minutes. The gel is incubated on an orbital shaker for one minute. These two steps are repeated a second time. The protein bands on the gel were stained with SimplyBlue® SafeStain. 20 ml of SimplyBlue is added to the gel and microwaved for one minute. For complete staining, the gel is incubated on an orbital shaker overnight. De-staining of the gels in water was then performed to reduce background and bring out the intensity of the bands of interest.

iii. SEC-MALS

This method combines separation of proteins using HPLC size-exclusion chromatography (SEC) with simultaneous detection using UV, multi-angle laser light scattering, and refractive index. A Tosoh TSKgel G2000SWXL, 5 μm, 125 Å 7.8 mm×30 cm (Tosoh Product number: 08540), HPLC column was used. The Mobile Phase Buffer System (pH 7.0) consisted of: 400 mM NaCl; 20 mM MOPS; 20 mM a-D Methyl Mannopyranoside; 0.1 mM MnCl₂; and 0.1 mM CaCl₂. The HPLC was run under the following conditions: Temperature: Room Temperature; Flow Rate: 1 ml/min; ConA Concentration: Between 1 mg/ml and 3 mg/ml; sample Injection size: 100 μl

The following detection equipment was used: a Hitachi L-4250 UV-Vis Detector with detection performed at 280 nm; a Wyatt miniDAWN MALS Detector with detection performed at 685 nm; and a Wyatt OptiLab rEX Refractive Index Detector with detection at 660 nm or 690 nm

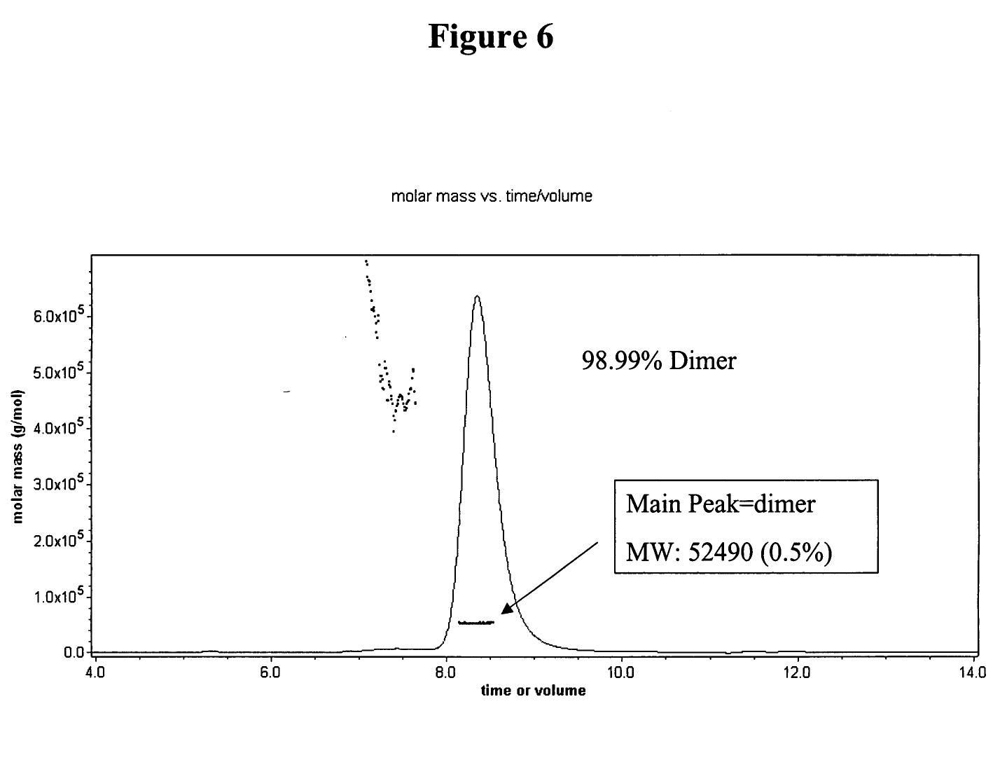

FIG. 6 is a graphical depiction of the SEC-MALS (size-exclusion chromatography equipped with multiangle light scattering) characterization showing that pET32, the quad mutant ConA (D58N, N118C, H121C, and E192Q) is a stable dimer of high purity (˜98%). The figure depicts both the UV trace (solid line) with the molar mass overlay (symbols) to show both purity of the pET 32 mutant ConA sample as well as the homogenous distribution of dimer within the primary peak. Peak integration results: 98.99% by relative peak area integration.

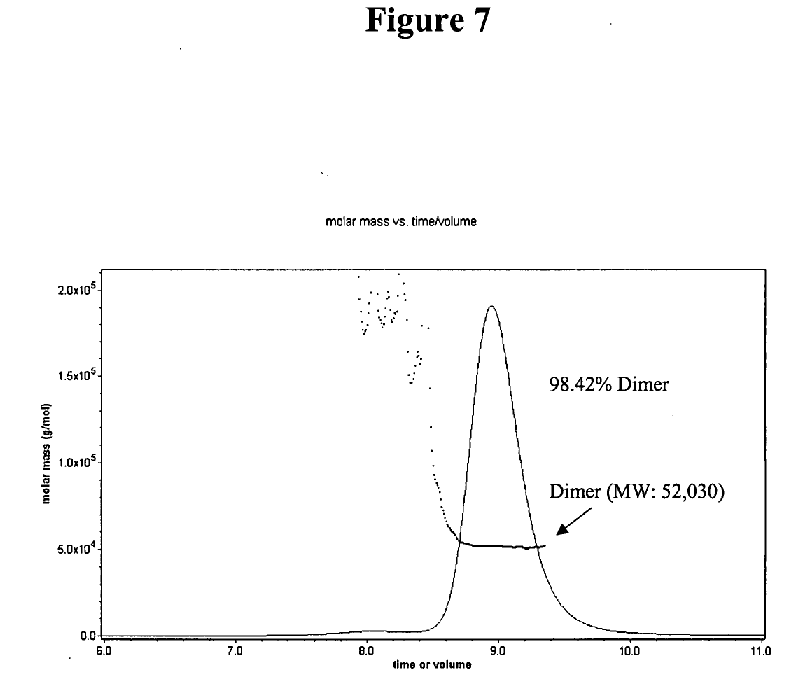

FIG. 7 is a graphical depiction of the SEC-MALS characterization showing that the quint mutant ConA (D58N, N118C, H121C, L142F and E192Q) is a stable dimer of high purity (˜98%). The figure depicts both the UV trace (solid line) with the molar mass overlay (symbols) to show both purity of the pET 32 quint mutant ConA sample as well as the homogenous distribution of dimer within the primary peak. Peak integration results: 98.42% purity by relative peak area integration. The L142F mutation was a PCR or mutation error. This L142F mutation did not effect the production of dimer.

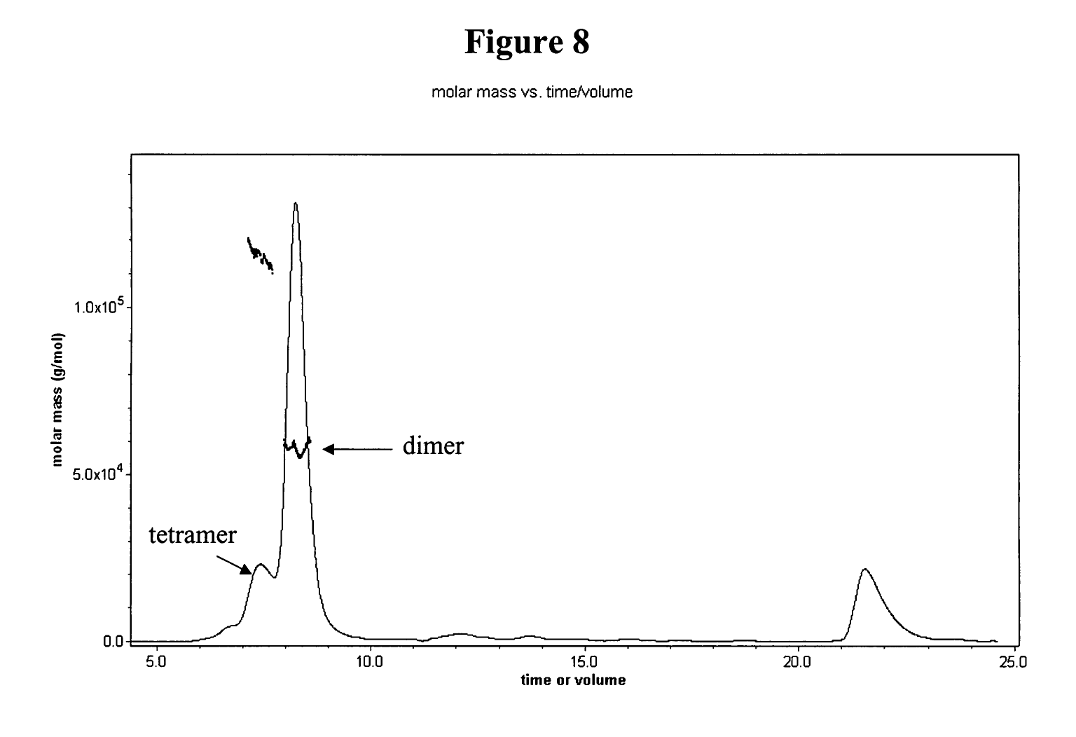

FIG. 8 is a representative graphical depiction of the SEC-MALS characterization of the ConA mutants (pET26, pET29, pET31, pET33) showing that pET26, a triple mutant ConA (G58N, N118C, E192Q) forms a dimer, but purifies as a mixture of dimer/tetramer with approximately 50-80% dimer. SEC-MALS was used to calculate the percent dimer purified for the ConA mutants that purified as a mixture of dimer/tetramer. The ratio of the area under the dimer peak versus the sum of the areas of all peaks present (total peak area) was calculated.

B. Functional Characterization of Mutant ConA

Functional properties of recombinant ConA have been characterized by Fluorescence Resonance Energy Transfer (FRET) using a PTI QuantaMaster fluorimeter. FRET occurs when two dye molecules interact in a distance-dependent fashion. The excitation energy from one dye (the donor) is transferred to a second dye molecule (the acceptor) without photon emission by an electrostatic dipole induced dipole interaction. This transfer of energy results in emission of the acceptor dye, which is one useful way to monitor the interaction of the proteins on which these two dyes reside.

i Affinity of Mutant ConA Using FRET

Mutant ConA was characterized by FRET. FIG. 12 is a graph of the results of a competition binding assay showing that the affinity of pET32 dimer ConA mutant (Kᵢ˜21 nM) is lower than a ConA tetramer (Kᵢ˜9.1 nM) by ˜two-fold. Tetrameric ConA was combined with HSA in a 384-well plate. Increasing concentration of competitor (either unlabeled tetramer or unlabeled dimer) was added in different wells to determine the amount of binding displaced (as indicated by changes in the ratio at wavelengths ˜600 nm and ˜700 nm). The EC₅₀ was calculated using a 4-parameter logistic equation. Kᵢ was estimated based on the concentration of labeled-tetramer used. The affinity of tetramer for HSA was determined independently using surface plasmon resonance.

ii. Dye Conjugations

Purified mutant ConA can be used in FRET interactions but must first be labeled with a fluorescent dye. Mutant ConA can be used as both the donor and the acceptor in FRET reactions, using both the Cy (Amersham) and Alexa (Molecular Probes) families of dyes. The conjugation reactions are similar regardless of which dye is used. Dimeric mutant ConA of the present invention allows for higher dye concentrations, thus increasing brightness.

Typically, 0.25 mg of dye was used to label 5 mg of mutant ConA. Purified wild-type ConA as well as pET 32 quad mutant ConA were used in conjugation reactions.

(a) FRET with Conjugated Mutant ConA

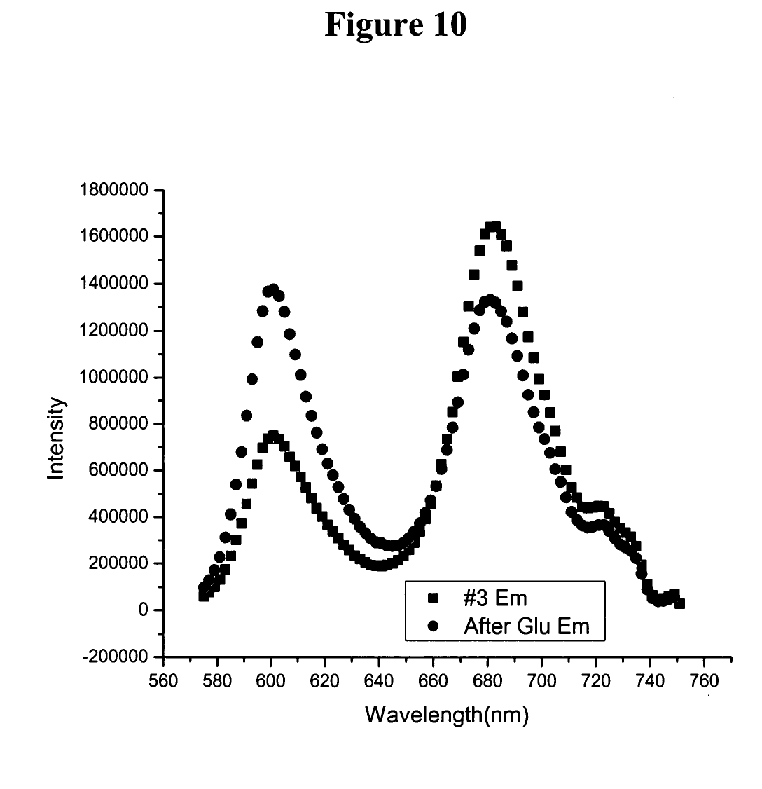

FRET was used to monitor the interaction of dye-labeled mutant ConA with dye and sugar-labeled therapeutic human serum albumin (tHSA). Mutant ConA was labeled with the donor, Cy3.5b. Therapeutic HSA was labeled with the acceptor, Alexa 647. Under these conditions, binding of Cy-labeled mutant ConA to Alexa-HSA when mixed in a ratio of 13 μM mutant ConA to 20 μM HSA, resulted in efficient FRET. FIG. 10 is a fluorescence emission spectra showing the FRET response upon the addition of glucose of pET32, the purified quad dimer mutant ConA labeled with Cy3.5b, combined with Alexa-labeled Human Serum Albumin (HSA). This figure illustrates the non-radiative transfer of energy from the donor (peak at ˜600 nm) to the acceptor (peak at ˜675 nm) in the presence of 500 mg/dL glucose (circles) and with no glucose (squares). The ratio of intensities at wavelengths ˜600 nm/675 nm is <1.0 before glucose addition and changes after the addition of glucose.

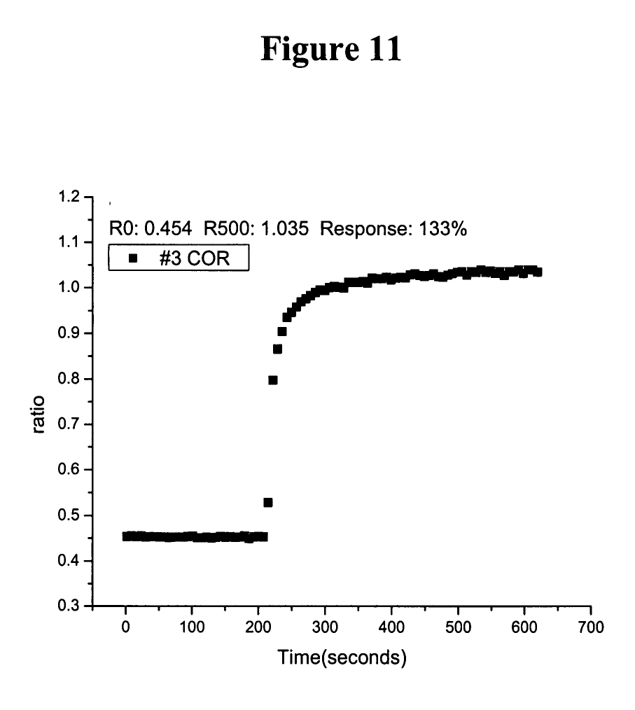

FIG. 11 is a time-based ratio scan of pET32, the purified quad dimer mutant ConA, labeled with Cy3.5b (donor) combined with Alexa-labeled HSA (acceptor). The ratio of intensities at ˜600 nm/˜675 nm is calculated and displayed over time after the addition of glucose. The spectra shows about 133% increase in response after the addition of glucose [(r₅₀₀−r₀)/r₀)×100=% increase in response].

(b) Sensors with Conjugated Mutant ConA

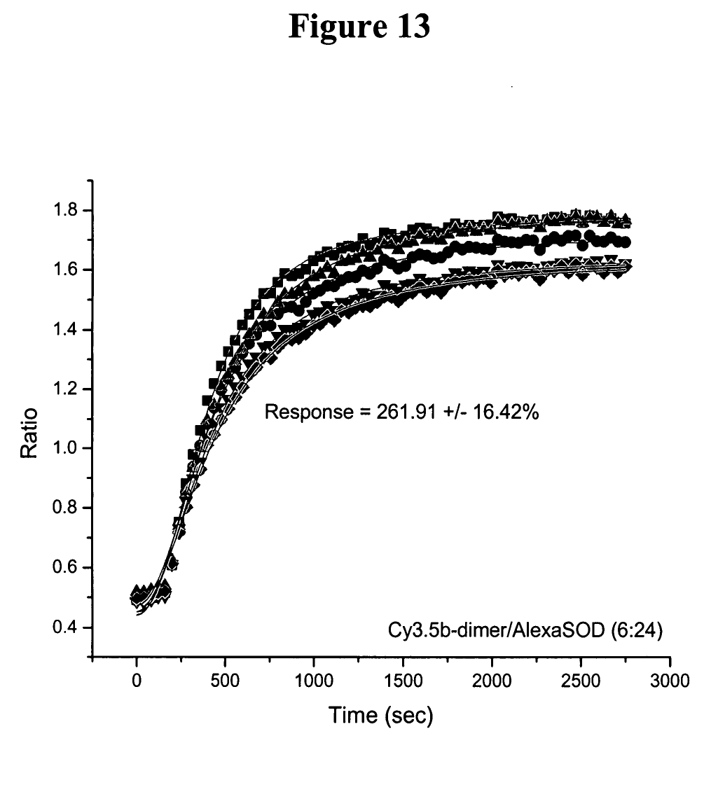

Sensors can be made with conjugated pairs of mutant ConA proteins of the present invention and HSA after they have been characterized in solution FRET. Examples of FRET-based sensors are described in U.S. Pat. No. 6,040,194, which has been incorporated by reference in its entirety. FIG. 13 is a fluorescence emission spectra showing the FRET response to the addition of 500 mg/dL glucose to sensors made with Cy3.5-labeled pET32 dimer mutant ConA (donor) and Alexa647-labeled superoxide dismutase (SOD) (acceptor) when mixed at a final concentration of 6 μM to 24 μM ratio. An approximately 262% response was obtained upon the addition of 500 mg/dL glucose.

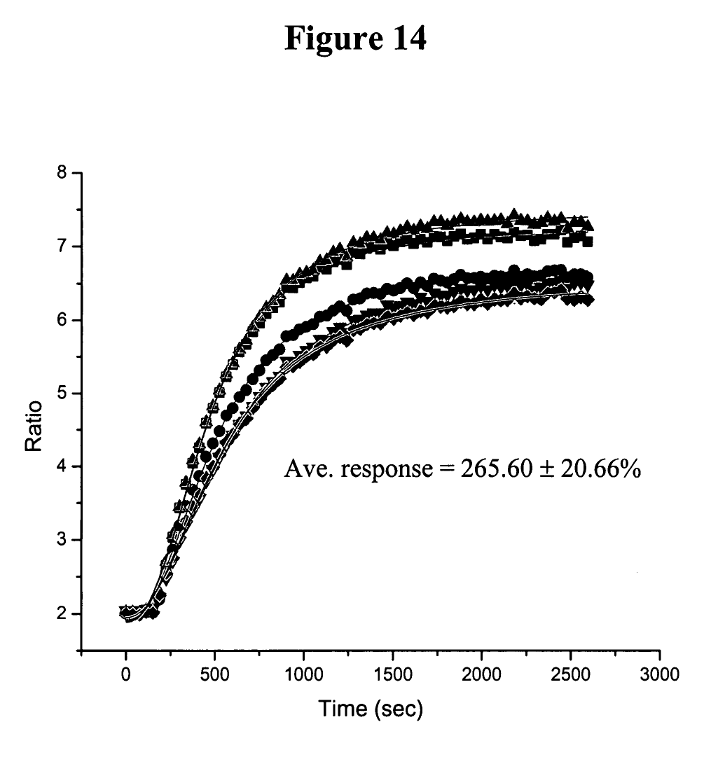

FIG. 14 is a fluorescence emission spectra showing the ˜266% FRET response to the addition of 500 mg/dL glucose to sensors made with Cy3.5-labeled pET32 dimer mutant ConA (donor) and Cy5.5-labeled superoxide dismutase (SOD) (acceptor). Sensors made with Cy3.5-labeled pET32 dimer mutant ConA and Cy5.5-labeled superoxide dismutase (SOD).

1. A purified mutant Concanavalin A (ConA) protein comprising the amino acid sequence of SEQ ID NO: 16, wherein said sequence comprises a substitution at amino acid residue 58 and a substitution at one or more of amino acid residue 118, amino acid residue 121, and amino acid residue 192, said purified mutant Con A having reduced dimer-dimer affinity compared to a corresponding wild type ConA protein.

2. The purified mutant ConA protein of claim 1, wherein an amino acid residue selected from the group consisting of asparagine, cysteine, proline, and glycine is substituted for the aspartic acid residue at position 58 of SEQ ID NO: 16.

3. The purified mutant ConA protein of claim 2, wherein an asparagine is substituted for the aspartic acid residue at position 58 of SEQ ID NO: 16.

4. The purified mutant ConA protein of claim 1, wherein an amino acid residue selected from the group of asparagine, cysteine, proline, glutamine, tyrosine, and glycine is substituted for the amino acid residue at one or more of position 118, 121, and 192 of SEQ ID NO: 16.

5. The purified mutant ConA protein of claim 1, wherein at least one of said substitutions replaces a naturally occurring amino acid residue with cysteine.

6. The purified mutant ConA protein of claim 1, wherein the protein comprises at least three substitutions.

7. The purified mutant ConA protein of claim 1, wherein the protein comprises at least four substitutions.

8. The purified mutant ConA protein of claim 1, said protein comprising a substitution at amino acid residue 58, amino acid residue 118, amino acid residue 121, and amino acid residue 192 of SEQ ID NO: 16.

9. The purified mutant ConA protein of claim 8, wherein a cysteine is substituted for the asparagine residue at position 118, a cysteine is substituted for the histidine residue at position 121, and a glutamine is substituted for the glutamic acid residue at position 192 of SEQ ID NO: 16.

10. The purified mutant ConA protein of claim 9, wherein an asparagine is substituted for the aspartic acid residue at position 58 of SEQ ID NO: 16.

11. The purified mutant ConA protein of claim 1, wherein the protein is substantially a dimer.

12. The purified mutant ConA protein of claim 1, wherein the protein is at least about 95% pure.

13. The purified mutant ConA protein of claim 1, wherein the protein exhibits glycoconjugate binding.

14. The purified mutant ConA protein of claim 1, wherein the protein further comprises a detectable label.

15. The purified mutant ConA protein of claim 14, wherein the label is selected from the group consisting of a radioactive label, a fluorescent label, an enzyme, a proximity-based signal generating label moiety, a homogeneous time resolved fluorescence (HTRF) component, and a luminescent oxygen channeling assay (LOCI) component.

16. A device capable of sensing a change in an amount of an analyte, the device comprising the purified mutant ConA protein of claim 1.

17. The device of claim 16, wherein at least a portion of the device is implantable.

18. The device of claim 16, wherein fluorescence can be used to detect the change in the amount of the analyte.

19. The device of claim 16, wherein the analyte comprises a carbohydrate selected from the group consisting of monosaccharides, disaccharides, polysaccharides or a combination thereof.

20. The device of claim 19, wherein the carbohydrate comprises glucose.

21. A purified mutant Concanavalin A (Con A) molecule, wherein the molecule comprises a polypeptide having an amino acid sequence selected from the group consisting of SEQ ID NOs: 6, 8, 10, 12, 14, 18, 20, 22, 24, and 26, or biologically active variants thereof.

22. A purified, isolated nucleic acid selected from the group consisting of SEQ ID NOs: 5, 7, 9, 11, 13, 17, 19, 21, 23, and 25.

23. A method of evaluating a carbohydrate in a sample comprising:
contacting the sample with a specific binding pair that comprises
(i) the purified mutant ConA protein of claim 1, and
(ii) a glycoconjugate, wherein the purified mutant ConA and glycoconjugate reversibly bind to each other; and
determining the extent to which carbohydrate present in the sample displaces glycoconjugate bound to the purified mutant ConA and reversibly binds to the purified mutant ConA.

24. The method of claim 23, wherein at least one of the purified mutant ConA protein and the glycoconjugate has a detectable label.

25. The method of claim 23, wherein the sample is selected from the group consisting of urine, blood, plasma, saliva, intracellular fluid, interstitial fluid, homogenized cells, and a cell extract.

26. The method of claim 23, wherein the glycoconjugate comprises a carbohydrate selected from the group consisting of monosaccharides, disaccharides, polysaccharides or a combination thereof.

27. The method of claim 26, wherein the carbohydrate comprises glucose.


SEQUENCE LISTING (56 sequences)

SEQ ID NO: 1 - DNA, 735 bp, Artificial
  SYNTHETIC
atggctaccgtagcgcaagctgctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttggtaagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagcaattcaacacatgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttgaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 2 - protein, 244 aa, Artificial
  SYNTHETIC
MATVAQAADTIVAVELDTYPNTDIGDPSYPHIGIDIKSVRSKKTAKWNMQNGKVGTAHIIYNSVGKRLSAVVSYPNADSATVSYDVDLDNVLPEWVRVGLSASTGLYKETNTILSWSFTSKLKSNSTHETNALHFMFNQFSKDQKDLILQGDATTGTDGNLELTRVSSNGSPQGSSVGRALFYAPVHIWESSAVVASFEATFTFLIKSPDSHPADGIAFFISNIDSSIPSGSTGRLLGLFPDAN

SEQ ID NO: 3 - protein, 237 aa, Canavalia ensiformis
ADTIVAVELDTYPNTDIGDPSYPHIGIDIKSVRSKKTAKWNMQNGKVGTAHIIYNSVDKRLSAVVSYPNADSATVSYDVDLDNVLPEWVRVGLSASTGLYKETNTILSWSFTSKLKSNSTHETNALHFMFNQFSKDQKDLILQGDATTGTEGNLRLTRVSSNGSPQGSSVGRALFYAPVHIWESSAVVASFEATFTFLIKSPDSHPADGIAFFISNIDSSIPSGSTGRLLGLFPDAN

SEQ ID NO: 4 - protein, 237 aa, Canavalia gladiata
ADTIVAVELDTYPNTDIGDPNYPHIGIDIKSVRSKKTAKWNMQNGKVGTAHIIYNSVGKRLSAVVSYPNGDSATVSYDVDLDNVLPEWVRVGLSASTGLYKETNTILSWSFTSKLKSNSTHETNALHFMFNQFSKDQKDLILQGDATTGTDGNLELTRVSSNGSPQGSSVGRALFYAPVHIWESSAVVASFDATFTFLIKSPDSHPADGIAFFISNIDSSIPSGSTGRLLGLFPDAN

SEQ ID NO: 5 - DNA, 735 bp, Artificial
  SYNTHETIC
atggctaccgtagcgcaagctgctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttaataagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagctgttcaacacatgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttcaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 6 - protein, 244 aa, Artificial
  SYNTHETIC
MATVAQAADTIVAVELDTYPNTDIGDPSYPHIGIDIKSVRSKKTAKWNMQNGKVGTAHIIYNSVNKRLSAVVSYPNADSATVSYDVDLDNVLPEWVRVGLSASTGLYKETNTILSWSFTSKLKSCSTHETNALHFMFNQFSKDQKDLILQGDATTGTDGNLELTRVSSNGSPQGSSVGRALFYAPVHIWESSAVVASFQATFTFLIKSPDSHPADGIAFFISNIDSSIPSGSTGRLLGLFPDAN

SEQ ID NO: 7 - DNA, 735 bp, Artificial
  SYNTHETIC
atggctaccgtagcgcaagctgctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttcctaagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagctgttcaacacatgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttcaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 8 - protein, 244 aa, Artificial
  SYNTHETIC
MATVAQAADTIVAVELDTYPNTDIGDPSYPHIGIDIKSVRSKKTAKWNMQNGKVGTAHIIYNSVPKRLSAVVSYPNADSATVSYDVDLDNVLPEWVRVGLSASTGLYKETNTILSWSFTSKLKSCSTHETNALHFMFNQFSKDQKDLILQGDATTGTDGNLELTRVSSNGSPQGSSVGRALFYAPVHIWESSAVVASFCATFTFLIKSPDSHPADGIAFFISNIDSSIPSGSTGRLLGLFPDAN

SEQ ID NO: 9 - DNA, 735 bp, Artificial
  SYNTHETIC
atggctaccgtagcgcaagctgctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttaataagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagctgttcaacatatgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttcaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 10 - protein, 244 aa, Artificial
  SYNTHETIC
MATVAQAADTIVAVELDTYPNTDIGDPSYPHIGIDIKSVRSKKTAKWNMQNGKVGTAHIIYNSVNKRLSAVVSYPNADSATVSYDVDLDNVLPEWVRVGLSASTGLYKETNTILSWSFTSKLKSCSTYETNALHFMFNQFSKDQKDLILQGDATTGTDGNLELTRVSSNGSPQGSSVGRALFYAPVHIWESSAVVASFQATFTFLIKSPDSHPADGIAFFISNIDSSIPSGSTGRLLGLFPDAN

SEQ ID NO: 11 - DNA, 735 bp, Artificial
  SYNTHETIC
atggctaccgtagcgcaagctgctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttaataagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagctgttcaacatgtgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttcaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 12 - protein, 244 aa, Artificial
  SYNTHETIC
MATVAQAADTIVAVELDTYPNTDIGDPSYPHIGIDIKSVRSKKTAKWNMQNGKVGTAHIIYNSVNKRLSAVVSYPNADSATVSYDVDLDNVLPEWVRVGLSASTGLYKETNTILSWSFTSKLKSCSTCETNALHFMFNQFSKDQKDLILQGDATTGTDGNLELTRVSSNGSPQGSSVGRALFYAPVHIWESSAVVASFQATFTFLIKSPDSHPADGIAFFISNIDSSIPSGSTGRLLGLFPDAN

SEQ ID NO: 13 - DNA, 735 bp, Artificial
  SYNTHETIC
atggctaccgtagcgcaagctgctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttaataagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagctgttcaacacctgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttcaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 14 - protein, 244 aa, Artificial
  SYNTHETIC
MATVAQAADTIVAVELDTYPNTDIGDPSYPHIGIDIKSVRSKKTAKWNMQNGKVGTAHIIYNSVNKRLSAVVSYPNADSATVSYDVDLDNVLPEWVRVGLSASTGLYKETNTILSWSFTSKLKSCSTPETNALHFMFNQFSKDQKDLILQGDATTGTDGNLELTRVSSNGSPQGSSVGRALFYAPVHIWESSAVVASFQATFTFLIKSPDSHPADGIAFFISNIDSSIPSGSTGRLLGLFPDAN

SEQ ID NO: 15 - DNA, 714 bp, Artificial
  SYNTHETIC
  misc_feature (61)..(63): n is a, c, g, or t
  misc_feature (208)..(210): n is a, c, g, or t
  misc_feature (385)..(387): n is a, c, g, or t
  misc_feature (451)..(453): n is a, c, g, or t
  misc_feature (463)..(465): n is a, c, g, or t
  misc_feature (502)..(504): n is a, c, g, or t
  misc_feature (604)..(606): n is a, c, g, or t
  misc_feature (622)..(624): n is a, c, g, or t
gctgataccattgtggcggtggaactggatacctatccgaacaccgatattggcgatccgnnntatccgcatattggcatcgatatcaaaagcgtgcgcagcaaaaaaaccgcgaaatggaacatgcagaacggtaaagttggcaccgcgcacatcatctataactctgttgataagagactaagtgctgttgtttcttatcctaacnnngactctgccactgtctcttacgacgttgacctcgacaatgtccttcctgaatgggttagagttggcctttctgcttcaaccggactttacaaagaaaccaataccattctctcatggtcttttacttctaagttgaagagcaattcaacacatgagacaaatgcactccatttcnnnttcaaccaatttagcaaagatcagaaggatttgatccttcaaggtgacgccacaacaggaacannnggtaacttgnnnctcacaagggtgtcaagtaatgggagtccacagggannnagtgtgggccgggctttgttctatgccccagtccacatttgggaaagttctgctgtggtggcaagctttgaagctacctttacatttctcataaaatcannngactctcacccagctnnnggaattgccttcttcatttcaaatattgacagttccatccctagtggttccactggaaggctccttggactcttccctgatgcaaattga

SEQ ID NO: 16 - protein, 237 aa, Artificial
  SYNTHETIC
  misc_feature (21)..(21): Xaa can be any naturally occurring amino acid
  misc_feature (70)..(70): Xaa can be any naturally occurring amino acid
  misc_feature (129)..(129): Xaa can be any naturally occurring amino acid
  misc_feature (151)..(151): Xaa can be any naturally occurring amino acid
  misc_feature (155)..(155): Xaa can be any naturally occurring amino acid
  misc_feature (168)..(168): Xaa can be any naturally occurring amino acid
  misc_feature (202)..(202): Xaa can be any naturally occurring amino acid
  misc_feature (208)..(208): Xaa can be any naturally occurring amino acid
ADTIVAVELDTYPNTDIGDPXYPHIGIDIKSVRSKKTAKWNMQNGKVGTAHIIYNSVDKRLSAVVSYPNXDSATVSYDVDLDNVLPEWVRVGLSASTGLYKETNTILSWSFTSKLKSNSTHETNALHFXFNQFSKDQKDLILQGDATTGTXGNLXLTRVSSNGSPQGXSVGRALFYAPVHIWESSAVVASFEATFTFLIKSXDSHPAXGIAFFISNIDSSIPSGSTGRLLGLFPDAN

SEQ ID NO: 17 - DNA, 713 bp, Artificial
  SYNTHETIC
ctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttaataagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagctgttcaacacatgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttcaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 18 - protein, 237 aa, Artificial
  SYNTHETIC
ADTIVAVELDTYPNTDIGDPSYPHIGIDIKSVRSKKTAKWNMQNGKVGTAHIIYNSVNKRLSAVVSYPNADSATVSYDVDLDNVLPEWVRVGLSASTGLYKETNTILSWSFTSKLKSCSTHETNALHFMFNQFSKDQKDLILQGDATTGTDGNLELTRVSSNGSPQGSSVGRALFYAPVHIWESSAVVASFQATFTFLIKSPDSHPADGIAFFISNIDSSIPSGSTGRLLGLFPDAN

SEQ ID NO: 19 - DNA, 714 bp, Artificial
  SYNTHETIC
gctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttcctaagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagctgttcaacacatgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttcaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 20 - protein, 237 aa, Artificial
  SYNTHETIC
ADTIVAVELDTYPNTDIGDPSYPHIGIDIKSVRSKKTAKWNMQNGKVGTAHIIYNSVPKRLSAVVSYPNADSATVSYDVDLDNVLPEWVRVGLSASTGLYKETNTILSWSFTSKLKSCSTHETNALHFMFNQFSKDQKDLILQGDATTGTDGNLELTRVSSNGSPQGSSVGRALFYAPVHIWESSAVVASFCATFTFLIKSPDSHPADGIAFFISNIDSSIPSGSTGRLLGLFPDAN

SEQ ID NO: 21 - DNA, 714 bp, Artificial
  SYNTHETIC
gctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttaataagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagctgttcaacatatgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttcaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 22 - protein, 237 aa, Artificial
  SYNTHETIC
ADTIVAVELDTYPNTDIGDPSYPHIGIDIKSVRSKKTAKWNMQNGKVGTAHIIYNSVNKRLSAVVSYPNADSATVSYDVDLDNVLPEWVRVGLSASTGLYKETNTILSWSFTSKLKSCSTYETNALHFMFNQFSKDQKDLILQGDATTGTDGNLELTRVSSNGSPQGSSVGRALFYAPVHIWESSAVVASFQATFTFLIKSPDSHPADGIAFFISNIDSSIPSGSTGRLLGLFPDAN

SEQ ID NO: 23 - DNA, 714 bp, Artificial
  SYNTHETIC
gctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttaataagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagctgttcaacatgtgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttcaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 24 - protein, 237 aa, Artificial
  SYNTHETIC
ADTIVAVELDTYPNTDIGDPSYPHIGIDIKSVRSKKTAKWNMQNGKVGTAHIIYNSVNKRLSAVVSYPNADSATVSYDVDLDNVLPEWVRVGLSASTGLYKETNTILSWSFTSKLKSCSTCETNALHFMFNQFSKDQKDLILQGDATTGTDGNLELTRVSSNGSPQGSSVGRALFYAPVHIWESSAVVASFQATFTFLIKSPDSHPADGIAFFISNIDSSIPSGSTGRLLGLFPDAN

SEQ ID NO: 25 - DNA, 714 bp, Artificial
  SYNTHETIC
gctgataccattgtggcggtggaactggatacctatccgaacaccgatattggcgatccgagctatccgcatattggcatcgatatcaaaagcgtgcgcagcaaaaaaaccgcgaaatggaacatgcagaacggtaaagttggcaccgcgcacatcatctataactctgttaataagagactaagtgctgttgtttcttatcctaacgctgactctgccactgtctcttacgacgttgacctcgacaatgtccttcctgaatgggttagagttggcctttctgcttcaaccggactttacaaagaaaccaataccattctctcatggtcttttacttctaagttgaagagctgttcaacacctgagacaaatgcactccatttcatgttcaaccaatttagcaaagatcagaaggatttgatccttcaaggtgacgccacaacaggaacagatggtaacttggaactcacaagggtgtcaagtaatgggagtccacagggaagcagtgtgggccgggctttgttctatgccccagtccacatttgggaaagttctgctgtggtggcaagctttcaagctacctttacatttctcataaaatcacccgactctcacccagctgatggaattgccttcttcatttcaaatattgacagttccatccctagtggttccactggaaggctccttggactcttccctgatgcaaattga

SEQ ID NO: 26 - protein, 237 aa, Artificial
  SYNTHETIC
ADTIVAVELDTYPNTDIGDPSYPHIGIDIKSVRSKKTAKWNMQNGKVGTAHIIYNSVNKRLSAVVSYPNADSATVSYDVDLDNVLPEWVRVGLSASTGLYKETNTILSWSFTSKLKSCSTPETNALHFMFNQFSKDQKDLILQGDATTGTDGNLELTRVSSNGSPQGSSVGRALFYAPVHIWESSAVVASFQATFTFLIKSPDSHPADGIAFFISNIDSSIPSGSTGRLLGLFPDAN

SEQ ID NO: 27 - DNA, 21 bp, Artificial
  SYNTHETIC
atggctaccgtagcgcaagct

SEQ ID NO: 28 - protein, 7 aa, Artificial
  SYNTHETIC
MATVAQA

SEQ ID NO: 29 - DNA, 20 bp, Artificial
  SYNTHETIC
attgtagcaagcagcactac

SEQ ID NO: 30 - DNA, 21 bp, Artificial
  SYNTHETIC
tagcaagcagcactactagtg

SEQ ID NO: 31 - DNA, 20 bp, Artificial
  SYNTHETIC
gcaagcagcactactagtga

SEQ ID NO: 32 - DNA, 24 bp, Artificial
  SYNTHETIC
gagattattatggtacatggatga

SEQ ID NO: 33 - DNA, 30 bp, Artificial
  SYNTHETIC
gccgatactattgttgctgttgaattggat

SEQ ID NO: 34 - DNA, 60 bp, Artificial
  SYNTHETIC
gaaatggagtgcatttgtctcatgtgttgaattgctcttcaacttagaagtaaaagacca

SEQ ID NO: 35 - DNA, 60 bp, Artificial
  SYNTHETIC
tggtcttttacttctaagttgaagagcaattcaacacatgagacaaatgcactccatttc

SEQ ID NO: 36 - DNA, 33 bp, Artificial
  SYNTHETIC
tcaatttgcatcagggaagagtccaaggagcct

SEQ ID NO: 37 - DNA, 60 bp, Artificial
  SYNTHETIC
cacatcatctataactctgtttgtaagagactaagtgctgttgtttcttatcctaacgct

SEQ ID NO: 38 - DNA, 60 bp, Artificial
  SYNTHETIC
agcgttaggataagaaacaacagcacttagtctcttacaaacagagttatagatgatgtg

SEQ ID NO: 39 - DNA, 60 bp, Artificial
  SYNTHETIC
cacatcatctataactctgttcctaagagactaagtgctgttgtttcttatcctaacgct

SEQ ID NO: 40 - DNA, 60 bp, Artificial
  SYNTHETIC
agcgttaggataagaaacaacagcacttagtctcttaggaacagagttatagatgatgtg

SEQ ID NO: 41 - DNA, 60 bp, Artificial
  SYNTHETIC
cacatcatctataactctgttaataagagactaagtgctgttgtttcttatcctaacgct

SEQ ID NO: 42 - DNA, 60 bp, Artificial
  SYNTHETIC
agcgttaggataagaaacaacagcacttagtctcttattaacagagttatagatgatgtg

SEQ ID NO: 43 - DNA, 60 bp, Artificial
  SYNTHETIC
tctgctgtggtggccagctttcaagctacctttacatttctcataaaatcacccgactct

SEQ ID NO: 44 - DNA, 60 bp, Artificial
  SYNTHETIC
agagtcgggtgattttatgagaaatgtaaaggtagcttgaaagctggccaccacagcaga

SEQ ID NO: 45 - DNA, 60 bp, Artificial
  SYNTHETIC
tctgctgtggtggccagctttccagctacctttacatttctcataaaatcacccgactct

SEQ ID NO: 46 - DNA, 60 bp, Artificial
  SYNTHETIC
agagtcgggtgattttatgagaaatgtaaaggtagctggaaagctggccaccacagcaga

SEQ ID NO: 47 - DNA, 60 bp, Artificial
  SYNTHETIC
tctgctgtggtggccagcttttgtgctacctttacatttctcataaaatcacccgactct

SEQ ID NO: 48 - DNA, 60 bp, Artificial
  SYNTHETIC
agagtcgggtgattttatgagaaatgtaaaggtagcacaaaagctggccaccacagcaga

SEQ ID NO: 49 - DNA, 60 bp, Artificial
  SYNTHETIC
tcatggtcttttacttctaagttgaagagctgttcaacacatgagacaaatgcactccat

SEQ ID NO: 50 - DNA, 60 bp, Artificial
  SYNTHETIC
atggagtgcatttgtctcatgtgttgaacagctcttcaacttagaagtaaaagaccatga

SEQ ID NO: 51 - DNA, 60 bp, Artificial
  SYNTHETIC
acttctaagttgaagagctgttcaacatatgagacaaatgcactccatttcatgttcaac

SEQ ID NO: 52 - DNA, 60 bp, Artificial
  SYNTHETIC
gttgaacatgaaatggagtgcatttgtctcatatgttgaacagctcttcaacttagaagt

SEQ ID NO: 53 - DNA, 60 bp, Artificial
  SYNTHETIC
acttctaagttgaagagctgttcaacatgtgagacaaatgcactccatttcatgttcaac

SEQ ID NO: 54 - DNA, 60 bp, Artificial
  SYNTHETIC
gttgaacatgaaatggagtgcatttgtctcacatgttgaacagctcttcaacttagaagt

SEQ ID NO: 55 - DNA, 59 bp, Artificial
  SYNTHETIC
acttctaagttgaagagctgttcaacacctgagacaaatgcactccatttcatgttcaa

SEQ ID NO: 56 - DNA, 60 bp, Artificial
  SYNTHETIC
gttgaacatgaaatggagtgcatttgtctcaggtgttgaacagctcttcaacttagaagt